SEQUENCE LISTING (86 sequences)

SEQ ID NO: 1 - DNA, 687 bp, Staphylococcus aureus
atgacagaatacttattaagtgctggcatatgtatggcaattgtttcaatattacttatagggatggctatcagtaatgtttcgaaagggcaatacgcaaagaggtttttctttttcgctactagttgcttagtgttaactttagttgtagtttcaagtctaagtagctcagcaaatgcatcacaaacagataacggcgtaaatagaagtggttctgaagatccaacagtatatagtgcaacttcaactaaaaaattacataaagaacctgcgacattaattaaagcgattgatggtgatacggttaaattaatgtacaaaggtcaaccaatgacattcagactattattagttgatacacctgaaacaaagcatcctaaaaaaggtgtagagaaatatggccctgaagcaagtgcatttacgaaaaaaatggtagaaaatgcaaataaaattgaagtcgagtttgacaaaggtcaaagaactgataaatatggacgtggcttagcgtatatttatgctgatggaaaaatggtaaacgaagctttagttcgtcaaggcttggctaaagttgcttatgtttataaacctaacaatacacatgaacaacttttaagaaaaagtgaagcacaagcgaaaaaagagaaattaaatatttggagcgaagacaacgctgattcaggtcaataa

SEQ ID NO: 2 - DNA, 612 bp, Artificial
  Nuc - Cloned DNA Sequence
aatgtttcgaaagggcaatacgcaaagaggtttttctttttcgctactagttgcttagtgttaactttagttgtagtttcaagtctaagtagctcagcaaatgcatcacaaacagataacggcgtaaatagaagtggttctgaagatccaacagtatatagtgcaacttcaactaaaaaattacataaagaacctgcgacattaattaaagcgattgatggtgatacggttaaattaatgtacaaaggtcaaccaatgacattcagactattattagttgatacacctgaaacaaagcatcctaaaaaaggtgtagagaaatatggccctgaagcaagtgcatttacgaaaaaaatggtagaaaatgcaaataaaattgaagtcgagtttgacaaaggtcaaagaactgataaatatggacgtggcttagcgtatatttatgctgatggaaaaatggtaaacgaagctttagttcgtcaaggcttggctaaagttgcttatgtttataaacctaacaatacacatgaacaacttttaagaaaaagtgaagcacaagcgaaaaaagagaaattaaatatttggagcgaagacaacgctgattcaggtcaataa

SEQ ID NO: 3 - protein, 228 aa, Staphylococcus aureus
MTEYLLSAGICMAIVSILLIGMAISNVSKGQYAKRFFFFATSCLVLTLVVVSSLSSSANASQTDNGVNRSGSEDPTVYSATSTKKLHKEPATLIKAIDGDTVKLMYKGQPMTFRLLLVDTPETKHPKKGVEKYGPEASAFTKKMVENANKIEVEFDKGQRTDKYGRGLAYIYADGKMVNEALVRQGLAKVAYVYKPNNTHEQLLRKSEAQAKKEKLNIWSEDNADSGQ

SEQ ID NO: 4 - protein, 203 aa, Artificial
  Nuc - Cloned peptide sequence
NVSKGQYAKRFFFFATSCLVLTLVVVSSLSSSANASQTDNGVNRSGSEDPTVYSATSTKKLHKEPATLIKAIDGDTVKLMYKGQPMTFRLLLVDTPETKHPKKGVEKYGPEASAFTKKMVENANKIEVEFDKGQRTDKYGRGLAYIYADGKMVNEALVRQGLAKVAYVYKPNNTHEQLLRKSEAQAKKEKLNIWSEDNADSGQ

SEQ ID NO: 5 - DNA, 1248 bp, Staphylococcus aureus
atgtcatatcattggtttaagaaaatgttactttcaacaagtattttaattttaagtagtagtagtttagggcttgcaacgcacacagttgaagcaaaggataacttaaatggagaaaaaccaactactaatttgaatcataatataacttcaccatcagtaaatagtgaaatgaataataatgagactgggacacctcacgaatcaaatcaaacgggtaatgaaggaacaggttcgaatagtcgtgatgctaatcctgattcgaataatgtgaagccagactcaaacaaccaaaacccaagtacagattcaaaaccagacccaaataaccaaaacccaagtccgaatccaaaaccagatccagataacccgaaaccaaaaccagacccagataaaccaaagccaaatctggatccaaaaccagatccagataacccgaaaccaaaaccagatccaaaaccagacccagataaaccaaagccaaatccggatccaaaaccagatccagataaaccaaagccaaatccgaatccaaaaccagaccctaataagccaaatcctaacccgtcaccagatcccgatcaacctggggattcaaatcattctggtggctcgaaaaatgggggaacatggaacccaaatgcttcagatggatctaatcaaggtcaatggcaaccaaatgggaatcaaggaaactcacaaaatcctactggtaatgattttgtatcccaacgatttttagccttggcaaatggggcttacaagtataatccgtatattttaaatcaaattaataagttgggcaaagattatggagaagttactgatgaagacatttataatattattcgaaaacaaaatttcagcggaaatgcatatttaaatggattacaacagcaatcgaattactttagattccaatatttcaatccattgaaatcagaaaggtactatcgtaatttagatgaacaagtactcgcattaattactggtgaaattggatcaatgccagatttgaaaaagcccgaagataagccggattcaaaacaacgctcatttgaaccgcatgaaaaagacgattttacagtagttaaaaaacaagaagataataagaaaagtgcgtcaactgcatatagtaaaagttggctagcaattgtatgttctatgatggtggtattttcaatcatgctattcttatttgtaaagcgaaataaaaagaaaaataaaaacgaatcacagcgacgataa

SEQ ID NO: 6 - DNA, 1149 bp, Artificial
  SdrH-like - Cloned DNA Sequence
gataacttaaatggagaaaaaccaactactaatttgaatcataatataacttcaccatcagtaaatagtgaaatgaataataatgagactgggacacctcacgaatcaaatcaaacgggtaatgaaggaacaggttcgaatagtcgtgatgctaatcctgattcgaataatgtgaagccagactcaaacaaccaaaacccaagtacagattcaaaaccagacccaaataaccaaaacccaagtccgaatccaaaaccagatccagataacccgaaaccaaaaccagacccagataaaccaaagccaaatctggatccaaaaccagatccagataacccgaaaccaaaaccagatccaaaaccagacccagataaaccaaagccaaatccggatccaaaaccagatccagataaaccaaagccaaatccgaatccaaaaccagaccctaataagccaaatcctaacccgtcaccagatcccgatcaacctggggattcaaatcattctggtggctcgaaaaatgggggaacatggaacccaaatgcttcagatggatctaatcaaggtcaatggcaaccaaatgggaatcaaggaaactcacaaaatcctactggtaatgattttgtatcccaacgatttttagccttggcaaatggggcttacaagtataatccgtatattttaaatcaaattaataagttgggcaaagattatggagaagttactgatgaagacatttataatattattcgaaaacaaaatttcagcggaaatgcatatttaaatggattacaacagcaatcgaattactttagattccaatatttcaatccattgaaatcagaaaggtactatcgtaatttagatgaacaagtactcgcattaattactggtgaaattggatcaatgccagatttgaaaaagcccgaagataagccggattcaaaacaacgctcatttgaaccgcatgaaaaagacgattttacagtagttaaaaaacaagaagataataagaaaagtgcgtcaactgcatatagtaaaagttggctagcaattgtatgttctatgatggtggtattttcaatcatgctattcttatttgtaaagcgaaataaaaagaaaaataaaaacgaatcacagcgacgataa

SEQ ID NO: 7 - protein, 415 aa, Staphylococcus aureus
MSYHWFKKMLLSTSILILSSSSLGLATHTVEAKDNLNGEKPTTNLNHNITSPSVNSEMNNNETGTPHESNQTGNEGTGSNSRDANPDSNNVKPDSNNQNPSTDSKPDPNNQNPSPNPKPDPDNPKPKPDPDKPKPNLDPKPDPDNPKPKPDPKPDPDKPKPNPDPKPDPDKPKPNPNPKPDPNKPNPNPSPDPDQPGDSNHSGGSKNGGTWNPNASDGSNQGQWQPNGNQGNSQNPTGNDFVSQRFLALANGAYKYNPYILNQINKLGKDYGEVTDEDIYNIIRKQNFSGNAYLNGLQQQSNYFRFQYFNPLKSERYYRNLDEQVLALITGEIGSMPDLKKPEDKPDSKQRSFEPHEKDDFTVVKKQEDNKKSASTAYSKSWLAIVCSMMVVFSIMLFLFVKRNKKKNKNESQRR

SEQ ID NO: 8 - protein, 382 aa, Artificial
  SdrH-like - Cloned peptide sequence
DNLNGEKPTTNLNHNITSPSVNSEMNNNETGTPHESNQTGNEGTGSNSRDANPDSNNVKPDSNNQNPSTDSKPDPNNQNPSPNPKPDPDNPKPKPDPDKPKPNLDPKPDPDNPKPKPDPKPDPDKPKPNPDPKPDPDKPKPNPNPKPDPNKPNPNPSPDPDQPGDSNHSGGSKNGGTWNPNASDGSNQGQWQPNGNQGNSQNPTGNDFVSQRFLALANGAYKYNPYILNQINKLGKDYGEVTDEDIYNIIRKQNFSGNAYLNGLQQQSNYFRFQYFNPLKSERYYRNLDEQVLALITGEIGSMPDLKKPEDKPDSKQRSFEPHEKDDFTVVKKQEDNKKSASTAYSKSWLAIVCSMMVVFSIMLFLFVKRNKKKNKNESQRR

SEQ ID NO: 9 - DNA, 1056 bp, Staphylococcus aureus
atgaaaaataaaaaacgtgttttaatagcgtcatcattatcatgtgcaattttattgttatcagcagcaacgactcaagcaaattcagctcataaagactcgcaagatcaaaataagaaagaacatgttgataagtctcaacaaaaagacaaacgtaatgttactaataaagataaaaattcaacagtacctgatgatattgggaaaaacggtaaaatcacaaaacgaactgaaacagtatatgatgagaaaacaaatatactccaaaatttacaattcgactttatcgatgatccaacttatgacaagaatgtattacttgttaaaaaacaaggctcaattcattcaaatttaaagtttgaatctcataaagaagaaaaaaattcaaattggttaaagtatccaagtgagtaccatgtagattttcaagtaaaaagaaatcgtaaaactgaaatattagaccaattgccgaaaaataaaatttcaactgcgaaagtagacagtacattttcatatagctcaggtggtaaattcgattcaacgaaaggtattggacgaacttcatcaaatagctactccaaaacgattagttataatcagcaaaattatgacacaattgccagcggtaaaaataataactggcatgtacactggtcagttattgcgaatgacttgaagtatggtggagaagtgaaaaatagaaatgatgaattattattctatagaaatacgagaattgctactgtagaaaaccctgaactaagctttgcttcaaaatatagatacccagcattagtaagaagtggctttaatccagaatttttaacttatttatctaatgaaaagtcaaatgagaaaacgcaatttgaagtaacatacacacgaaatcaagatattttgaaaaacagacctggaatacattatgcaccttcaattttagaaaaaaataaagatggtcaaaggttaattgtcacttatgaagttgattggaaaaataaaacagttaaagtcgttgataaatattctgatgacaataaaccttataaagaaggataa

SEQ ID NO: 10 - DNA, 972 bp, Artificial
  LukG - Cloned DNA Sequence
tcagctcataaagactcgcaagatcaaaataagaaagaacatgttgataagtctcaacaaaaagacaaacgtaatgttactaataaagataaaaattcaacagtacctgatgatattgggaaaaacggtaaaatcacaaaacgaactgaaacagtatatgatgagaaaacaaatatactccaaaatttacaattcgactttatcgatgatccaacttatgacaagaatgtattacttgttaaaaaacaaggctcaattcattcaaatttaaagtttgaatctcataaagaagaaaaaaattcaaattggttaaagtatccaagtgagtaccatgtagattttcaagtaaaaagaaatcgtaaaactgaaatattagaccaattgccgaaaaataaaatttcaactgcgaaagtagacagtacattttcatatagctcaggtggtaaattcgattcaacgaaaggtattggacgaacttcatcaaatagctactccaaaacgattagttataatcagcaaaattatgacacaattgccagcggtaaaaataataactggcatgtacactggtcagttattgcgaatgacttgaagtatggtggagaagtgaaaaatagaaatgatgaattattattctatagaaatacgagaattgctactgtagaaaaccctgaactaagctttgcttcaaaatatagatacccagcattagtaagaagtggctttaatccagaatttttaacttatttatctaatgaaaagtcaaatgagaaaacgcaatttgaagtaacatacacacgaaatcaagatattttgaaaaacagacctggaatacattatgcaccttcaattttagaaaaaaataaagatggtcaaaggttaattgtcacttatgaagttgattggaaaaataaaacagttaaagtcgttgataaatattctgatgacaataaaccttataaagaaggataa

SEQ ID NO: 11 - protein, 351 aa, Staphylococcus aureus
MKNKKRVLIASSLSCAILLLSAATTQANSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPSILEKNKDGQRLIVTYEVDWKNKTVKVVDKYSDDNKPYKEG

SEQ ID NO: 12 - protein, 323 aa, Artificial
  LukG - Cloned peptide sequence
SAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPSILEKNKDGQRLIVTYEVDWKNKTVKVVDKYSDDNKPYKEG

SEQ ID NO: 13 - DNA, 1017 bp, Staphylococcus aureus
atgattaaacaattatacaaaaacatcacaatttgtagtttagcaatatctactgcattaactgtatttccggcaacttcttatgcaaaaattaattctgaaattaaagctgtttctgagaagaatcttgatggtgatactaaaatgtatacacgtacagctacaacaagtgatagtcaaaaaaatattactcaaagcttacaatttaatttcttaactgaacctaattatgataaagaaacagtatttattaaagcaaaaggtacaattggtagtggtttgagaattttagacccaaatggttattggaatagtacattaagatggcctggatcttattcagtttcaattcaaaatgttgatgacaacaacaatacaaatgtgactgactttgcaccaaaaaatcaggatgaatcaagagaagttaaatatacgtatggttataaaacaggtggagatttttcgattaatcgtggaggcttaactggaaatattacaaaagagagtaattattcagagacgattagttatcaacaaccatcatatcgtacattacttgatcaatctacgtcacataaaggtgtaggttggaaagtagaagcacatttgataaataatatgggacatgaccatacgagacaattaactaatgatagtgataatagaactaaaagtgaaatcttttctttaacacgaaatggaaatttatgggcgaaagataatttcacacctaaagacaaaatgcctgtaactgtgtctgaagggtttaatccagaatttttagctgttatgtcacatgataaaaaagacaaaggtaaatcacaatttgttgttcattataaaagatcaatggatgagtttaaaatagattggaatcgccatggtttctggggctattggtctggtgaaaaccatgtagataaaaaagaagaaaaattatcagcattatatgaagttgattggaagacacatgatgtgaagtttgtaaaagtacttaatgataatgaaaagaaataa

SEQ ID NO: 14 - DNA, 927 bp, Artificial
  LukH - Cloned DNA Sequence
attaattctgaaattaaagctgtttctgagaagaatcttgatggtgatactaaaatgtatacacgtacagctacaacaagtgatagtcaaaaaaatattactcaaagcttacaatttaatttcttaactgaacctaattatgataaagaaacagtatttattaaagcaaaaggtacaattggtagtggtttgagaattttagacccaaatggttattggaatagtacattaagatggcctggatcttattcagtttcaattcaaaatgttgatgacaacaacaatacaaatgtgactgactttgcaccaaaaaatcaggatgaatcaagagaagttaaatatacgtatggttataaaacaggtggagatttttcgattaatcgtggaggcttaactggaaatattacaaaagagagtaattattcagagacgattagttatcaacaaccatcatatcgtacattacttgatcaatctacgtcacataaaggtgtaggttggaaagtagaagcacatttgataaataatatgggacatgaccatacgagacaattaactaatgatagtgataatagaactaaaagtgaaatcttttctttaacacgaaatggaaatttatgggcgaaagataatttcacacctaaagacaaaatgcctgtaactgtgtctgaagggtttaatccagaatttttagctgttatgtcacatgataaaaaagacaaaggtaaatcacaatttgttgttcattataaaagatcaatggatgagtttaaaatagattggaatcgccatggtttctggggctattggtctggtgaaaaccatgtagataaaaaagaagaaaaattatcagcattatatgaagttgattggaagacacatgatgtgaagtttgtaaaagtacttaatgataatgaaaagaaataa

SEQ ID NO: 15 - protein, 338 aa, Staphylococcus aureus
MIKQLYKNITICSLAISTALTVFPATSYAKINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHDVKFVKVLNDNEKK

SEQ ID NO: 16 - protein, 308 aa, Artificial
  LukH - Cloned peptide sequence
INSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHDVKFVKVLNDNEKK

SEQ ID NO: 17 - DNA, 930 bp, Staphylococcus aureus
atgaaaaaattagtacctttattattagccttattacttctagttgctgcatgtggtactggtggtaaacaaagcagtgataagtcaaatggcaaattaaaagtagtaacgacgaattcaattttatatgatatggctaaaaatgttggtggagacaacgtcgatattcatagtattgtacctgttggtcaagatcctcatgaatatgaagttaaacctaaagatattaaaaagttaactgacgctgacgttattttatacaacggattaaatttagagactggtaacggttggtttgaaaaagccttagaacaggctggtaaatcattaaaagataaaaaagttatcgcagtatcaaaagatgttaaacctatctatttaaacggtgaagaaggcaacaaagataaacaagatccacacgcatggttaagtttagataatggtattaaatacgtaaaaacaattcaacaaacatttatcgataacgacaaaaaacataaagcagattatgaaaagcaaggtaacaaatacattgctcaattggaaaaattaaataatgacagtaaagacaaatttaatgacattccaaaagaacaacgtgccatgattacaagtgaaggtgccttcaagtacttctcaaaacaatacggtattacaccaggttatatttgggaaattaacactgaaaaacaaggtacacctgaacaaatgagacaagctattgagtttgttaaaaagcacaaattaaaacacttattagtagaaacaagtgttgataagaaagcaatggaaagtttatctgaagaaacgaagaaagatatctttggtgaagtgtacacagattcaatcggtaaagaaggcactaaaggtgactcttactacaaaatgatgaaatcaaatattgaaactgtacacggaagcatgaaataa

SEQ ID NO: 18 - DNA, 831 bp, Artificial
  MntC - Cloned DNA Sequence
aaagtagtaacgacgaattcaattttatatgatatggctaaaaatgttggtggagacaacgtcgatattcatagtattgtacctgttggtcaagatcctcatgaatatgaagttaaacctaaagatattaaaaagttaactgacgctgacgttattttatacaacggattaaatttagagactggtaacggttggtttgaaaaagccttagaacaggctggtaaatcattaaaagataaaaaagttatcgcagtatcaaaagatgttaaacctatctatttaaacggtgaagaaggcaacaaagataaacaagatccacacgcatggttaagtttagataatggtattaaatacgtaaaaacaattcaacaaacatttatcgataacgacaaaaaacataaagcagattatgaaaagcaaggtaacaaatacattgctcaattggaaaaattaaataatgacagtaaagacaaatttaatgacattccaaaagaacaacgtgccatgattacaagtgaaggtgccttcaagtacttctcaaaacaatacggtattacaccaggttatatttgggaaattaacactgaaaaacaaggtacacctgaacaaatgagacaagctattgagtttgttaaaaagcacaaattaaaacacttattagtagaaacaagtgttgataagaaagcaatggaaagtttatctgaagaaacgaagaaagatatctttggtgaagtgtacacagattcaatcggtaaagaaggcactaaaggtgactcttactacaaaatgatgaaatcaaatattgaaactgtacacggaagcatgaaataa

SEQ ID NO: 19 - protein, 309 aa, Staphylococcus aureus
MKKLVPLLLALLLLVAACGTGGKQSSDKSNGKLKVVTTNSILYDMAKNVGGDNVDIHSIVPVGQDPHEYEVKPKDIKKLTDADVILYNGLNLETGNGWFEKALEQAGKSLKDKKVIAVSKDVKPIYLNGEEGNKDKQDPHAWLSLDNGIKYVKTIQQTFIDNDKKHKADYEKQGNKYIAQLEKLNNDSKDKFNDIPKEQRAMITSEGAFKYFSKQYGITPGYIWEINTEKQGTPEQMRQAIEFVKKHKLKHLLVETSVDKKAMESLSEETKKDIFGEVYTDSIGKEGTKGDSYYKMMKSNIETVHGSMK

SEQ ID NO: 20 - protein, 276 aa, Artificial
  MntC - Cloned peptide sequence
KVVTTNSILYDMAKNVGGDNVDIHSIVPVGQDPHEYEVKPKDIKKLTDADVILYNGLNLETGNGWFEKALEQAGKSLKDKKVIAVSKDVKPIYLNGEEGNKDKQDPHAWLSLDNGIKYVKTIQQTFIDNDKKHKADYEKQGNKYIAQLEKLNNDSKDKFNDIPKEQRAMITSEGAFKYFSKQYGITPGYIWEINTEKQGTPEQMRQAIEFVKKHKLKHLLVETSVDKKAMESLSEETKKDIFGEVYTDSIGKEGTKGDSYYKMMKSNIETVHGSMK

SEQ ID NO: 21 - DNA, 2076 bp, Staphylococcus aureus
atgttaagaggacaagaagaaagaaagtatagtattagaaagtattcaataggcgtggtgtcagtgttagcggctacaatgtttgttgtgtcatcacatgaagcacaagcctcggaaaaaacaccaacgtccaatgcagcggcacaaaaagaaacactaaatcaaccgggagaacaagggaacgcgataacgtcacatcaaatgcagtcaggaaagcaattagacgatatgcataaagagaatggtaaaagtggaacagtgacagaaggtaaagacacgcttcaatcatcgaagcatcaatcaacacaaaatagtaaaacaatcagaacgcaaaatgataatcaagtaaagcaagattctgaacgacaaggttctaaacagtcacaccaaaataatgcgactaataatactgaacgtcaaaatgatcaggttcaaaatacccatcatgctgaacgtaatggatcacaatcgacaacgtcacaatcgaatgatgttgataaatcacaaccatccattccggcacaaaaggtattacccaatcatgataaagcagcaccaacttcaactacacccccgtctaatgataaaactgcacctaaatcaacaaaagcacaagatgcaaccacggacaaacatccaaatcaacaagatacacatcaacccgcgcatcaaatcatagatgcaaagcaagatgatactgttcgccaaagtgaacagaaaccacaagttggcgatttaagtaaacatatcgatggtcaaaattccccagagaaaccgacagataaaaatactgataataaacaactaatcaaagatgcgcttcaagcgcctaaaacacgttcgactacaaatgcagcagcagatgctaaaaaggttcgaccacttaaagcgaatcaagtacaaccacttaacaaatatccagttgtttttgtacatggatttttaggattagtaggcgataatgcacctgctttatatccaaattattggggtggaaataaatttaaagttatcgaggaattgagaaagcaaggctataatgtacatcaagcaagtgtaagtgcatttggtagtaactatgatcgcgctgtagaactttattattacattaaaggtggtcgcgtagattatggcgcagcacatgcagctaaatacggacatgagcgctatggtaagacttataaaggaatcatgcctaattgggaacctggtaaaaaggtacatcttgtagggcatagtatgggtggtcaaacaattcgtttaatggaagagtttttaagaaatggtaacaaagaagaaattgcctatcataaagcgcatggtggagaaatatcaccattattcactggtggtcataacaatatggttgcatcaatcacaacattagcaacaccacataatggttcacaagcagctgataagtttggaaatacagaagctgttagaaaaatcatgttcgctttaaatcgatttatgggtaacaagtattcgaatatcgatttaggattaacgcaatggggctttaaacaattaccaaatgagagttacattgactatataaaacgcgttagtaaaagcaaaatttggacatcagacgataatgctgcctatgatttaacgttagatggctctgcaaaattgaacaacatgacaagtatgaatcctaatattacgtatacgacttatacaggtgtgtcttcacatactggtccattaggttatgaaaatcctgatttaggtacatttttcttaatggatacaacgagtagaattattggtcatgatgcaagagaagaatggcgtaaaaatgatggtgtcgtaccagtgatttcgtcgttacatccatccaatcaaccatttattaatgttacgaatgatgaacctgccacacgcagaggtatctggcaagttaaaccaatcatacaaggatgggatcatgtcgattttatcggtgtggacttcctggatttcaaacgtaaaggtgcagaacttgccaacttctatacaggtattataaatgacttgttgcgtgtggaagcgactgaaagtaaaggaacacaattgaaagcaagttaa

SEQ ID NO: 22 - DNA, 1962 bp, Artificial
  Lip2 - Cloned DNA Sequence
gaaaaaacaccaacgtccaatgcagcggcacaaaaagaaacactaaatcaaccgggagaacaagggaacgcgataacgtcacatcaaatgcagtcaggaaagcaattagacgatatgcataaagagaatggtaaaagtggaacagtgacagaaggtaaagacacgcttcaatcatcgaagcatcaatcaacacaaaatagtaaaacaatcagaacgcaaaatgataatcaagtaaagcaagattctgaacgacaaggttctaaacagtcacaccaaaataatgcgactaataatactgaacgtcaaaatgatcaggttcaaaatacccatcatgctgaacgtaatggatcacaatcgacaacgtcacaatcgaatgatgttgataaatcacaaccatccattccggcacaaaaggtattacccaatcatgataaagcagcaccaacttcaactacacccccgtctaatgataaaactgcacctaaatcaacaaaagcacaagatgcaaccacggacaaacatccaaatcaacaagatacacatcaacccgcgcatcaaatcatagatgcaaagcaagatgatactgttcgccaaagtgaacagaaaccacaagttggcgatttaagtaaacatatcgatggtcaaaattccccagagaaaccgacagataaaaatactgataataaacaactaatcaaagatgcgcttcaagcgcctaaaacacgttcgactacaaatgcagcagcagatgctaaaaaggttcgaccacttaaagcgaatcaagtacaaccacttaacaaatatccagttgtttttgtacatggatttttaggattagtaggcgataatgcacctgctttatatccaaattattggggtggaaataaatttaaagttatcgaggaattgagaaagcaaggctataatgtacatcaagcaagtgtaagtgcatttggtagtaactatgatcgcgctgtagaactttattattacattaaaggtggtcgcgtagattatggcgcagcacatgcagctaaatacggacatgagcgctatggtaagacttataaaggaatcatgcctaattgggaacctggtaaaaaggtacatcttgtagggcatagtatgggtggtcaaacaattcgtttaatggaagagtttttaagaaatggtaacaaagaagaaattgcctatcataaagcgcatggtggagaaatatcaccattattcactggtggtcataacaatatggttgcatcaatcacaacattagcaacaccacataatggttcacaagcagctgataagtttggaaatacagaagctgttagaaaaatcatgttcgctttaaatcgatttatgggtaacaagtattcgaatatcgatttaggattaacgcaatggggctttaaacaattaccaaatgagagttacattgactatataaaacgcgttagtaaaagcaaaatttggacatcagacgataatgctgcctatgatttaacgttagatggctctgcaaaattgaacaacatgacaagtatgaatcctaatattacgtatacgacttatacaggtgtgtcttcacatactggtccattaggttatgaaaatcctgatttaggtacatttttcttaatggatacaacgagtagaattattggtcatgatgcaagagaagaatggcgtaaaaatgatggtgtcgtaccagtgatttcgtcgttacatccatccaatcaaccatttattaatgttacgaatgatgaacctgccacacgcagaggtatctggcaagttaaaccaatcatacaaggatgggatcatgtcgattttatcggtgtggacttcctggatttcaaacgtaaaggtgcagaacttgccaacttctatacaggtattataaatgacttgttgcgtgtggaagcgactgaaagtaaaggaacacaattgaaagcaagttaa

SEQ ID NO: 23 - protein, 691 aa, Staphylococcus aureus
MLRGQEERKYSIRKYSIGVVSVLAATMFVVSSHEAQASEKTPTSNAAAQKETLNQPGEQGNAITSHQMQSGKQLDDMHKENGKSGTVTEGKDTLQSSKHQSTQNSKTIRTQNDNQVKQDSERQGSKQSHQNNATNNTERQNDQVQNTHHAERNGSQSTTSQSNDVDKSQPSIPAQKVLPNHDKAAPTSTTPPSNDKTAPKSTKAQDATTDKHPNQQDTHQPAHQIIDAKQDDTVRQSEQKPQVGDLSKHIDGQNSPEKPTDKNTDNKQLIKDALQAPKTRSTTNAAADAKKVRPLKANQVQPLNKYPVVFVHGFLGLVGDNAPALYPNYWGGNKFKVIEELRKQGYNVHQASVSAFGSNYDRAVELYYYIKGGRVDYGAAHAAKYGHERYGKTYKGIMPNWEPGKKVHLVGHSMGGQTIRLMEEFLRNGNKEEIAYHKAHGGEISPLFTGGHNNMVASITTLATPHNGSQAADKFGNTEAVRKIMFALNRFMGNKYSNIDLGLTQWGFKQLPNESYIDYIKRVSKSKIWTSDDNAAYDLTLDGSAKLNNMTSMNPNITYTTYTGVSSHTGPLGYENPDLGTFFLMDTTSRIIGHDAREEWRKNDGVVPVISSLHPSNQPFINVTNDEPATRRGIWQVKPIIQGWDHVDFIGVDFLDFKRKGAELANFYTGIINDLLRVEATESKGTQLKAS

SEQ ID NO: 24 - protein, 653 aa, Artificial
  Lip2 - Cloned peptide sequence
EKTPTSNAAAQKETLNQPGEQGNAITSHQMQSGKQLDDMHKENGKSGTVTEGKDTLQSSKHQSTQNSKTIRTQNDNQVKQDSERQGSKQSHQNNATNNTERQNDQVQNTHHAERNGSQSTTSQSNDVDKSQPSIPAQKVLPNHDKAAPTSTTPPSNDKTAPKSTKAQDATTDKHPNQQDTHQPAHQIIDAKQDDTVRQSEQKPQVGDLSKHIDGQNSPEKPTDKNTDNKQLIKDALQAPKTRSTTNAAADAKKVRPLKANQVQPLNKYPVVFVHGFLGLVGDNAPALYPNYWGGNKFKVIEELRKQGYNVHQASVSAFGSNYDRAVELYYYIKGGRVDYGAAHAAKYGHERYGKTYKGIMPNWEPGKKVHLVGHSMGGQTIRLMEEFLRNGNKEEIAYHKAHGGEISPLFTGGHNNMVASITTLATPHNGSQAADKFGNTEAVRKIMFALNRFMGNKYSNIDLGLTQWGFKQLPNESYIDYIKRVSKSKIWTSDDNAAYDLTLDGSAKLNNMTSMNPNITYTTYTGVSSHTGPLGYENPDLGTFFLMDTTSRIIGHDAREEWRKNDGVVPVISSLHPSNQPFINVTNDEPATRRGIWQVKPIIQGWDHVDFIGVDFLDFKRKGAELANFYTGIINDLLRVEATESKGTQLKAS

SEQ ID NO: 25 - DNA, 1488 bp, Staphylococcus aureus
atgagagaaaattttaaattacgtaaaatgaaagtcggtttagtatcagttgcaattactatgttatatatcatgacaaacggacaagctgaagcatctgaaactaatcagaaggtaagtacaaatcaagaatcaaaagctgtttcgcagactgaacaaaattccaaagaaacaaaagcggcagagtcttctaaaaactttgtaaatttagatcctatcaaaccaggagctcaaaaagtaacaggtactacattaccgaatcatattattttacttaatatagatggtaaaagtgctgattcagttgaaggaggatatggtgattttataactgcaaatgacaaaggagagtttgagtatcctttaaataatcgtaaaattgtccataaccaagaaattgaggtatcatcatcaagccctgatttaggtgacgatgaagaggatgaagaagtagaagaatcttcaactgaaaaagctggtgctgaggaagaaagtacagatgctaaagctacatatacaacaccacgatatgaaaaagcgtatgaaataccgaaagaacaactaaaagaaaaagatggacaccaccaagttttcatcgaaccaattactgaaggttcaggtattattaaaggtcatacgtctgtaaaaggtaaagttgctctatctattaataataaatttattaattttgaagaaagagctaaaggtggaattagtaaagaagatactaaagctagttccgatggtgtttggatgcctattaatgaaaaaggatattttgattttgatttcaaaaaaaatccttttgataacttagagttaaagaaaaatgatgaaatctcattaacatttgcacctgatgatgaagatgaagcattgaagtcattaattttcaaaactaaagtaacgagcttagaagatatcgataaagcagaaacaaaatatgaccatactaaagtggaaaaagtaaaagtattgaaagatgttaaagaagatatacatgtagatgaaatttacggaagcttatatcatacagaaaaaggtaaaggtattcttgataaacaaggaactaaagagattacagggaagactaaattcgcgaatgcagtagtgaaagtatattctgacttaggtgatgcgcaattgttccctgatattcaagtagatgaaaatggtaaatttagctttgatgctgaaaaagctggtttcagattgcaaaatggagaaacattgaattttgcagtagttaaacctattactggtgacctattacatcaaggatttgtctctaaatatattgatgtatatgaatctccggaagaaaagaaagaacgtgaatttgaagaaaaacttgaaaacacgcctgcatatcataaattacatggtgataaaattgtaggatatgatgttcaaggtaatccatcaacttggttctatccattaggtgaaaagaaagttgaacgtacaacacctaaattagaaaaataa

SEQ ID NO: 26 - DNA, 1392 bp, Artificial
  putative - Cloned DNA Sequence
tctgaaactaatcagaaggtaagtacaaatcaagaatcaaaagctgtttcgcagactgaacaaaattccaaagaaacaaaagcggcagagtcttctaaaaactttgtaaatttagatcctatcaaaccaggagctcaaaaagtaacaggtactacattaccgaatcatattattttacttaatatagatggtaaaagtgctgattcagttgaaggaggatatggtgattttataactgcaaatgacaaaggagagtttgagtatcctttaaataatcgtaaaattgtccataaccaagaaattgaggtatcatcatcaagccctgatttaggtgacgatgaagaggatgaagaagtagaagaatcttcaactgaaaaagctggtgctgaggaagaaagtacagatgctaaagctacatatacaacaccacgatatgaaaaagcgtatgaaataccgaaagaacaactaaaagaaaaagatggacaccaccaagttttcatcgaaccaattactgaaggttcaggtattattaaaggtcatacgtctgtaaaaggtaaagttgctctatctattaataataaatttattaattttgaagaaagagctaaaggtggaattagtaaagaagatactaaagctagttccgatggtgtttggatgcctattaatgaaaaaggatattttgattttgatttcaaaaaaaatccttttgataacttagagttaaagaaaaatgatgaaatctcattaacatttgcacctgatgatgaagatgaagcattgaagtcattaattttcaaaactaaagtaacgagcttagaagatatcgataaagcagaaacaaaatatgaccatactaaagtggaaaaagtaaaagtattgaaagatgttaaagaagatatacatgtagatgaaatttacggaagcttatatcatacagaaaaaggtaaaggtattcttgataaacaaggaactaaagagattacagggaagactaaattcgcgaatgcagtagtgaaagtatattctgacttaggtgatgcgcaattgttccctgatattcaagtagatgaaaatggtaaatttagctttgatgctgaaaaagctggtttcagattgcaaaatggagaaacattgaattttgcagtagttaaacctattactggtgacctattacatcaaggatttgtctctaaatatattgatgtatatgaatctccggaagaaaagaaagaacgtgaatttgaagaaaaacttgaaaacacgcctgcatatcataaattacatggtgataaaattgtaggatatgatgttcaaggtaatccatcaacttggttctatccattaggtgaaaagaaagttgaacgtacaacacctaaattagaaaaataa

SEQ ID NO: 27 - protein, 495 aa, Staphylococcus aureus
MRENFKLRKMKVGLVSVAITMLYIMTNGQAEASETNQKVSTNQESKAVSQTEQNSKETKAAESSKNFVNLDPIKPGAQKVTGTTLPNHIILLNIDGKSADSVEGGYGDFITANDKGEFEYPLNNRKIVHNQEIEVSSSSPDLGDDEEDEEVEESSTEKAGAEEESTDAKATYTTPRYEKAYEIPKEQLKEKDGHHQVFIEPITEGSGIIKGHTSVKGKVALSINNKFINFEERAKGGISKEDTKASSDGVWMPINEKGYFDFDFKKNPFDNLELKKNDEISLTFAPDDEDEALKSLIFKTKVTSLEDIDKAETKYDHTKVEKVKVLKDVKEDIHVDEIYGSLYHTEKGKGILDKQGTKEITGKTKFANAVVKVYSDLGDAQLFPDIQVDENGKFSFDAEKAGFRLQNGETLNFAVVKPITGDLLHQGFVSKYIDVYESPEEKKEREFEEKLENTPAYHKLHGDKIVGYDVQGNPSTWFYPLGEKKVERTTPKLEK

SEQ ID NO: 28 - protein, 463 aa, Artificial
  putative - Cloned peptide sequence
SETNQKVSTNQESKAVSQTEQNSKETKAAESSKNFVNLDPIKPGAQKVTGTTLPNHIILLNIDGKSADSVEGGYGDFITANDKGEFEYPLNNRKIVHNQEIEVSSSSPDLGDDEEDEEVEESSTEKAGAEEESTDAKATYTTPRYEKAYEIPKEQLKEKDGHHQVFIEPITEGSGIIKGHTSVKGKVALSINNKFINFEERAKGGISKEDTKASSDGVWMPINEKGYFDFDFKKNPFDNLELKKNDEISLTFAPDDEDEALKSLIFKTKVTSLEDIDKAETKYDHTKVEKVKVLKDVKEDIHVDEIYGSLYHTEKGKGILDKQGTKEITGKTKFANAVVKVYSDLGDAQLFPDIQVDENGKFSFDAEKAGFRLQNGETLNFAVVKPITGDLLHQGFVSKYIDVYESPEEKKEREFEEKLENTPAYHKLHGDKIVGYDVQGNPSTWFYPLGEKKVERTTPKLEK

SEQ ID NO: 29 - DNA, 3747 bp, Staphylococcus aureus
atggcgaaaaaattcaattacaaactaccatcaatggttgcattaacgcttgtaggttcagcagtcactgcacatcaagttcaagcagctgagacgacacaagatcaaactactaataaaaatgttttagatagtaataaagttaaagcaactactgaacaagcaaaagctgaggtaaaaaatccaacgcaaaacatttctggcactcaagtatatcaagaccctgctattgtccaaccaaaaacagcaaataacaaaacaggcaatgctcaagtaagtcaaaaagttgatactgcacaagtaaatggtgacactcgtgctaatcaatcagcgactacaaataatacgcagcctgttgcaaagtcaacaagcactacagcacctaaaactaacactaatgttacaaatgctggttatagtttagttgatgatgaagatgataattcagaacatcaaattaatccagaattaattaaatcagctgctaaacctgcagctcttgaaacgcaatataaagccgcagcacctaaagctaaaactgaagcgacacctaaagtaactacttttagcgcttcagcacaaccaagatcagttgctgcaacaccaaaaacgagtttgccaaaatataaaccacaagtaaactcttcaattaacgattacattcgtaaaaataacttaaaagcacctaaaattgaagaagattatacatcttacttccctaaatacgcataccgtaacggcgtaggtcgtcctgaaggtatcgtagttcatgatacagctaatgatcgttcgacgataaatggtgaaattagttatatgaaaaataactatcaaaacgcattcgtacatgcatttgttgatggggatcgtataatcgaaacagcaccaacggattacttatcttggggtgtcggtgcagtcggtaaccctagattcatcaatgttgaaatcgtacacacacacgactatgcttcatttgcacgttcaatgaataactatgctgactatgcagctacacaattacaatattatggtttaaaaccagacagtgctgagtatgatggaaatggtacagtatggactcactacgctgtaagtaaatatttaggtggtacggaccatgccgatccacatggatatttaagaagtcataattatagttatgatcaattatatgacttaattaatgaaaaatatttaataaaaatgggtaaagtggcgccatggggtacgcaatttacaactacccctactacaccatcaaaaccaacaacaccgtcgaaaccatcaactggtaaattaacagttgcagcaaacaatggtgtcgcacaaatcaaaccaacaaatagtggtttatatactactgtttacgacaaaactggtaaagcaactaatgaagttcaaaaaacatttgctgtatctaaaacagctacattaggtaatcaaaaattctatcttgttcaagattacaattctggtaataaatttggttgggttaaagaaggcgatgtggtttacaacacagctaaatcacctgtaaatgtaaatcaatcatattcaatcaaatctggtacgaaactttatacagtaccttggggtacatctaaacaagttgctggtagcgtgtctggctctggaaaccaaacatttaaggcttcaaagcaacaacaaattgataaatcaatttatttatatggctctgtgaatggtaaatctggttgggtaagtaaagcatatttagttgatactgctaaacctacgcctacaccaatacctaagccatcaacacctacaacaaataataaattaacagtttcatcattaaacggtgttgctcaaattaatgctaaaaacaatggcttattcactacagtttatgacaaaactggtaagccaacgaaagaagttcaaaaaacatttgctgtaacaaaagaagcaagtttaggtggaaacaaattctacttagttaaagattacaatagtccaactttaattggttgggttaaacaaggtgacgttatttataacaatgcaaaatcacctgtaaatgtaatgcaaacatatacagtaaaaccaggcactaaattatattcagtaccttggggcacttataaacaagaagctggtgcagtttctggtacaggtaaccaaacttttaaagcgactaagcaacaacaaattgataaatctatctatttatttggaactgtaaatggtaaatctggttgggtaagtaaagcatatttagctgtacctgctgcacctaaaaaagcagtagcacaaccaaaaacagctgtaaaagcttatactgttactaaaccacaaacgactcaaacagttagcaagattgctcaagttaaaccaaacaacactggtattcgtgcttctgtttatgaaaaaacagcgaaaaacggtgcgaaatatgcagaccgtacgttctatgtaacaaaagagcgtgctcatggtaatgaaacgtatgtattattaaacaatacaagccataacatcccattaggttggttcaatgtaaaagacttaaatgttcaaaacctaggcaaagaagttaaaacgactcaaaaatatactgttaataaatcaaataacggcttatcaatggttccttggggtactaaaaaccaagtcattttaacaggcaataacattgctcaaggtacatttaatgcaacgaaacaagtatctgtaggcaaagatgtttatttatacggtactattaataaccgcactggttgggtaaatgcaaaagatttaactgcaccaactgctgtgaaaccaactacatcagctgccaaagattataactacacttatgtaattaaaaatggtaatggttattactatgtaacaccaaattctgatacagctaaatactcattaaaagcatttaatgaacaaccattcgcagttgttaaagaacaagtcattaatggacaaacttggtactatggtaaattatctaacggtaaattagcatggattaaatcaactgatttagctaaagaattaattaagtataatcaaacaggtatgacattaaaccaagttgctcaaatacaagctggtttacaatataaaccacaagtacaacgtgtaccaggtaagtggacagatgctaactttaatgatgttaagcatgcaatggatacgaagcgtttagctcaagatccagcattaaaatatcaattcttacgcttagaccaaccacaaaatatttctattgataaaattaatcaattcttaaaaggtaaaggtgtattagaaaaccaaggtgctgcatttaacaaagctgctcaaatgtatggcattaatgaagtttatcttatctcacatgccctattagaaacaggtaacggtacttctcaattagcgaaaggtgcagatgtagtgaacaacaaagttgtaactaactcaaacacgaaataccataacgtatttggtattgctgcatatgataacgatcctttacgtgaaggtattaaatatgctaaacaagctggttgggacacagtatcaaaagcaatcgttggtggtgctaaattcatcggcaactcatatgtaaaagctggtcaaaatacactttacaaaatgagatggaatcctgcacatccaggaacacaccaatatgctacagatgtagattgggctaacatcaatgctaaaatcatcaaaggctactatgataaaattggcgaagtcggcaaatacttcgacatcccacaatataaataa

SEQ ID NO: 30 - DNA, 576 bp, Artificial
  Atl - Cloned DNA Sequence
tcaaaaccaacaacaccgtcgaaaccatcaactggtaaattaacagttgcagcaaacaatggtgtcgcacaaatcaaaccaacaaatagtggtttatatactactgtttacgacaaaactggtaaagcaactaatgaagttcaaaaaacatttgctgtatctaaaacagctacattaggtaatcaaaaattctatcttgttcaagattacaattctggtaataaatttggttgggttaaagaaggcgatgtggtttacaacacagctaaatcacctgtaaatgtaaatcaatcatattcaatcaaatctggtacgaaactttatacagtaccttggggtacatctaaacaagttgctggtagcgtgtctggctctggaaaccaaacatttaaggcttcaaagcaacaacaaattgataaatcaatttatttatatggctctgtgaatggtaaatctggttgggtaagtaaagcatatttagttgatactgctaaacctacgcctacaccaatacctaagccatcaacacctacaacaaataataaattaacagtttcatcattaaacggtaaataa

SEQ ID NO: 31 - protein, 1248 aa, Staphylococcus aureus
MAKKFNYKLPSMVALTLVGSAVTAHQVQAAETTQDQTTNKNVLDSNKVKATTEQAKAEVKNPTQNISGTQVYQDPAIVQPKTANNKTGNAQVSQKVDTAQVNGDTRANQSATTNNTQPVAKSTSTTAPKTNTNVTNAGYSLVDDEDDNSEHQINPELIKSAAKPAALETQYKAAAPKAKTEATPKVTTFSASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQFTTTPTTPSKPTTPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKSGTKLYTVPWGTSKQVAGSVSGSGNQTFKASKQQQIDKSIYLYGSVNGKSGWVSKAYLVDTAKPTPTPIPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYTVTKPQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTINNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 32 - protein, 191 aa, Artificial
  Atl - Cloned peptide sequence
SKPTTPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKSGTKLYTVPWGTSKQVAGSVSGSGNQTFKASKQQQIDKSIYLYGSVNGKSGWVSKAYLVDTAKPTPTPIPKPSTPTTNNKLTVSSLNGK

SEQ ID NO: 33 - DNA, 435 bp, Staphylococcus aureus
atgaaattaaaatcatttataactgtaactttggcactgggcatgatcgcaacgactggcgctactgtgacaagtaatgaggtatctgcagcagaaaaggacaaactaccggcaactcaaaaagctaaagaaatgcaaaatgttccatatacaattgcagtagatggcattatggctttcaatcaatcttacttaaatttaccaaaagatagccaattatcatatttagatttaggaaataaagttaaagctttattatatgatgaacgtggtgtaacacctgagaagattcgaaatgcaaaatctgccgtttacacgattacttggaaagatggtagtaaaaaagaagtggatcttaagaaagatagctacacagcaaacttgtttgattcaaattcaattaaacaaattgatattaatgtaaaaactaaataa

SEQ ID NO: 34 - DNA, 339 bp, Artificial
  Hypothetical - Cloned DNA Sequence
aaggacaaactaccggcaactcaaaaagctaaagaaatgcaaaatgttccatatacaattgcagtagatggcattatggctttcaatcaatcttacttaaatttaccaaaagatagccaattatcatatttagatttaggaaataaagttaaagctttattatatgatgaacgtggtgtaacacctgagaagattcgaaatgcaaaatctgccgtttacacgattacttggaaagatggtagtaaaaaagaagtggatcttaagaaagatagctacacagcaaacttgtttgattcaaattcaattaaacaaattgatattaatgtaaaaactaaataa

SEQ ID NO: 35 - protein, 144 aa, Staphylococcus aureus
MKLKSFITVTLALGMIATTGATVTSNEVSAAEKDKLPATQKAKEMQNVPYTIAVDGIMAFNQSYLNLPKDSQLSYLDLGNKVKALLYDERGVTPEKIRNAKSAVYTITWKDGSKKEVDLKKDSYTANLFDSNSIKQIDINVKTK

SEQ ID NO: 36 - protein, 112 aa, Artificial
  Hypothetical - Cloned peptide sequence
KDKLPATQKAKEMQNVPYTIAVDGIMAFNQSYLNLPKDSQLSYLDLGNKVKALLYDERGVTPEKIRNAKSAVYTITWKDGSKKEVDLKKDSYTANLFDSNSIKQIDINVKTK

SEQ ID NO: 37 - DNA, 2007 bp, Staphylococcus aureus
atgaaaaagataaaaattgttccacttattttaatagttgtagttgtcgggtttggtatatatttttatgcttccaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtacaagatgataccttcgttccacttaaaaccgttaaaaaaatggatgaatatttaagtgatttcgcaaaaaaatttcatcttacaactaatgaaacagaaagtcgtaactatcctctaggaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataa

SEQ ID NO: 38 - DNA, 1932 bp, Artificial
  Pbp2a - Cloned DNA Sequence
aaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtacaagatgataccttcgttccacttaaaaccgttaaaaaaatggatgaatatttaagtgatttcgcaaaaaaatttcatcttacaactaatgaaacagaaagtcgtaactatcctctaggaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataa

SEQ ID NO: 39 - protein, 668 aa, Staphylococcus aureus
MKKIKIVPLILIVVVVGFGIYFYASKDKEINNTIDAIEDKNFKQVYKDSSYISKSDNGEVEMTERPIKIYNSLGVKDINIQDRKIKKVSKNKKRVDAQYKIKTNYGNIDRNVQFNFVKEDGMWKLDWDHSVIIPGMQKDQSIHIENLKSERGKILDRNNVELANTGTAYEIGIVPKNVSKKDYKAIAKELSISEDYIKQQMDQNWVQDDTFVPLKTVKKMDEYLSDFAKKFHLTTNETESRNYPLGKATSHLLGYVGPINSEELKQKEYKGYKDDAVIGKKGLEKLYDKKLQHEDGYRVTIVDDNSNTIAHTLIEKKKKDGKDIQLTIDAKVQKSIYNNMKNDYGSGTAIHPQTGELLALVSTPSYDVYPFMYGMSNEEYNKLTEDKKEPLLNKFQITTSPGSTQKILTAMIGLNNKTLDDKTSYKIDGKGWQKDKSWGGYNVTRYEVVNGNIDLKQAIESSDNIFFARVALELGSKKFEKGMKKLGVGEDIPSDYPFYNAQISNKNLDNEILLADSGYGQGEILINPVQILSIYSALENNGNINAPHLLKDTKNKVWKKNIISKENINLLTDGMQQVVNKTHKEDIYRSYANLIGKSGTAELKMKQGETGRQIGWFISYDKDNPNMMMAINVKDVQDKGMASYNAKISGKVYDELYENGNKKYDIDE

SEQ ID NO: 40 - protein, 643 aa, Artificial
  Pbp2a - Cloned peptide sequence
KDKEINNTIDAIEDKNFKQVYKDSSYISKSDNGEVEMTERPIKIYNSLGVKDINIQDRKIKKVSKNKKRVDAQYKIKTNYGNIDRNVQFNFVKEDGMWKLDWDHSVIIPGMQKDQSIHIENLKSERGKILDRNNVELANTGTAYEIGIVPKNVSKKDYKAIAKELSISEDYIKQQMDQNWVQDDTFVPLKTVKKMDEYLSDFAKKFHLTTNETESRNYPLGKATSHLLGYVGPINSEELKQKEYKGYKDDAVIGKKGLEKLYDKKLQHEDGYRVTIVDDNSNTIAHTLIEKKKKDGKDIQLTIDAKVQKSIYNNMKNDYGSGTAIHPQTGELLALVSTPSYDVYPFMYGMSNEEYNKLTEDKKEPLLNKFQITTSPGSTQKILTAMIGLNNKTLDDKTSYKIDGKGWQKDKSWGGYNVTRYEVVNGNIDLKQAIESSDNIFFARVALELGSKKFEKGMKKLGVGEDIPSDYPFYNAQISNKNLDNEILLADSGYGQGEILINPVQILSIYSALENNGNINAPHLLKDTKNKVWKKNIISKENINLLTDGMQQVVNKTHKEDIYRSYANLIGKSGTAELKMKQGETGRQIGWFISYDKDNPNMMMAINVKDVQDKGMASYNAKISGKVYDELYENGNKKYDIDE

SEQ ID NO: 41 - DNA, 1029 bp, Staphylococcus aureus
atgaaaggtaaatttttaaaagttagttctttattcgttgcaactttgacaacagcgacacttgtgagttctccagcagcaaacgcgttatcatcaaaagctatggacaatcatccacaacaaacgcagtcaagcaaacagcaaacacctaagattaaaaaaggcggtaaccttaaaccattagaacaacgtgaacacgcaaatgttatattaccgaataacgatcgtcaccaaatcacagatacaacgaatggtcattatgcacccgtaacttatattcaagttgaagcacctactggtacatttattgcttccggtgtagttgtaggtaaagatactcttttaacaaataaacacgtcgtagatgctacgcacggtgatcctcatgctttaaaagcattcccttctgcaattaaccaagacaattatccaaatggtggtttcactgctgaacaaatcactaaatattcaggcgaaggtgatttagcaatagttaaattctcccctaatgagcaaaacaaacatattggtgaagtagttaaaccagcaacaatgagtaataatgctgaaacacaagttaaccaaaatattactgtaacaggatatcctggtgataaacctgtagcaacaatgtgggaaagtaaaggaaaaatcacttacctcaaaggcgaagctatgcaatatgatttaagtacaactggtggtaactcaggttcacctgtatttaatgaaaaaaatgaagtgatcggaattcattggggcggtgtaccaaatgaatttaatggtgcggtatttattaatgaaaatgtacgcaacttcttaaaacaaaatattgaagatatccattttgccaacgatgaccaacctaataacccagataatcctgataaccctaacaatcctgataaccctaacaatcctgataaccctaacaacccagatgaaccaaataaccctgacaaccctaacaaccctgataatccagacaatggcgataacaataattcagacaaccctgacgctgcataa

SEQ ID NO: 42 - DNA, 936 bp, Artificial
  SspA - Cloned DNA Sequence
tcatcaaaagctatggacaatcatccacaacaaacgcagtcaagcaaacagcaaacacctaagattaaaaaaggcggtaaccttaaaccattagaacaacgtgaacacgcaaatgttatattaccgaataacgatcgtcaccaaatcacagatacaacgaatggtcattatgcacccgtaacttatattcaagttgaagcacctactggtacatttattgcttccggtgtagttgtaggtaaagatactcttttaacaaataaacacgtcgtagatgctacgcacggtgatcctcatgctttaaaagcattcccttctgcaattaaccaagacaattatccaaatggtggtttcactgctgaacaaatcactaaatattcaggcgaaggtgatttagcaatagttaaattctcccctaatgagcaaaacaaacatattggtgaagtagttaaaccagcaacaatgagtaataatgctgaaacacaagttaaccaaaatattactgtaacaggatatcctggtgataaacctgtagcaacaatgtgggaaagtaaaggaaaaatcacttacctcaaaggcgaagctatgcaatatgatttaagtacaactggtggtaactcaggttcacctgtatttaatgaaaaaaatgaagtgatcggaattcattggggcggtgtaccaaatgaatttaatggtgcggtatttattaatgaaaatgtacgcaacttcttaaaacaaaatattgaagatatccattttgccaacgatgaccaacctaataacccagataatcctgataaccctaacaatcctgataaccctaacaatcctgataaccctaacaacccagatgaaccaaataaccctgacaaccctaacaaccctgataatccagacaatggcgataacaataattcagacaaccctgacgctgca

SEQ ID NO: 43 - protein, 342 aa, Staphylococcus aureus
MKGKFLKVSSLFVATLTTATLVSSPAANALSSKAMDNHPQQTQSSKQQTPKIKKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 44 - protein, 312 aa, Artificial
  SspA - Cloned peptide sequence
SSKAMDNHPQQTQSSKQQTPKIKKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 45 - DNA, 492 bp, Staphylococcus aureus
atgctcaaaagaagtttattatttttaactgttttattgttattattctcattttcttcaattactaatgaggtaagtgcatcaagttcattcgacaaaggaaaatataaaaaaggcgatgacgcgagttattttgaaccaacaggcccgtatttgatggtaaatgtgactggagttgatggtaaaggaaatgaattgctatcccctcattatgtcgagtttcctattaaacctgggactacacttacaaaagaaaaaattgaatactatgtcgaatgggcattagatgcgacagcatataaagagtttagagtagttgaattagatccaagcgcaaagatcgaagtcacttattatgataagaataagaaaaaagaagaaacgaagtctttccctataacagaaaaaggttttgttgtcccagatttatcagagcatattaaaaaccctggattcaacttaattacaaaggttgttatagaaaagaaataa

SEQ ID NO: 46 - DNA, 408 bp, Artificial
  Sak - Cloned DNA Sequence
agttcattcgacaaaggaaaatataaaaaaggcgatgacgcgagttattttgaaccaacaggcccgtatttgatggtaaatgtgactggagttgatggtaaaggaaatgaattgctatcccctcattatgtcgagtttcctattaaacctgggactacacttacaaaagaaaaaattgaatactatgtcgaatgggcattagatgcgacagcatataaagagtttagagtagttgaattagatccaagcgcaaagatcgaagtcacttattatgataagaataagaaaaaagaagaaacgaagtctttccctataacagaaaaaggttttgttgtcccagatttatcagagcatattaaaaaccctggattcaacttaattacaaaggttgttatagaaaagaaataa

SEQ ID NO: 47 - protein, 163 aa, Staphylococcus aureus
MLKRSLLFLTVLLLLFSFSSITNEVSASSSFDKGKYKKGDDASYFEPTGPYLMVNVTGVDGKGNELLSPHYVEFPIKPGTTLTKEKIEYYVEWALDATAYKEFRVVELDPSAKIEVTYYDKNKKKEETKSFPITEKGFVVPDLSEHIKNPGFNLITKVVIEKK

SEQ ID NO: 48 - protein, 135 aa, Artificial
  Sak - Cloned peptide sequence
SSFDKGKYKKGDDASYFEPTGPYLMVNVTGVDGKGNELLSPHYVEFPIKPGTTLTKEKIEYYVEWALDATAYKEFRVVELDPSAKIEVTYYDKNKKKEETKSFPITEKGFVVPDLSEHIKNPGFNLITKVVIEKK

SEQ ID NO: 49 - DNA, 702 bp, Staphylococcus aureus
atgaaaaagacaattatggcatcatcattagcagtggcattaggtgtaacaggttacgcagcaggtacaggacatcaagcacacgctgctgaagtaaacgttgatcaagcacacttagttgacttagcgcataatcaccaagatcaattaaatgcagctccaatcaaagatggtgcatatgacatccactttgtaaaagatggtttccaatataactttacttcaaatggtactacatggtcatggagctatgaagcagctaatggtcaaactgctggtttctcaaacgttgcaggtgcagactacactacttcatacaaccaaggttcagatgtacaatcagtaagctacaatgcacaatcaagtaactcaaacgttgaagctgtttcagctccaacttaccataactacagcacttcaactacttcaagttcagtgagattaagcaatggtaatactgcaggtgctactggttcatcagcagctcaaatcatggctcaacgtactggtgtttcagcttctacatgggctgcaatcatcgctcgtgaatcaaatggtcaagtaaatgcttacaacccatcaggtgcttcaggtttattccaaactatgccaggttggggtccgacaaacactgttgaccaacaaatcaacgcagctgttaaagcatacaaagcacaaggtttaggtgcttggggattctaa

SEQ ID NO: 50 - DNA, 609 bp, Artificial
  IsaA - Cloned DNA Sequence
gtaaacgttgatcaagcacacttagttgacttagcgcataatcaccaagatcaattaaatgcagctccaatcaaagatggtgcatatgacatccactttgtaaaagatggtttccaatataactttacttcaaatggtactacatggtcatggagctatgaagcagctaatggtcaaactgctggtttctcaaacgttgcaggtgcagactacactacttcatacaaccaaggttcagatgtacaatcagtaagctacaatgcacaatcaagtaactcaaacgttgaagctgtttcagctccaacttaccataactacagcacttcaactacttcaagttcagtgagattaagcaatggtaatactgcaggtgctactggttcatcagcagctcaaatcatggctcaacgtactggtgtttcagcttctacatgggctgcaatcatcgctcgtgaatcaaatggtcaagtaaatgcttacaacccatcaggtgcttcaggtttattccaaactatgccaggttggggtccgacaaacactgttgaccaacaaatcaacgcagctgttaaagcatacaaagcacaaggtttaggtgcttggggattctaa

SEQ ID NO: 51 - protein, 233 aa, Staphylococcus aureus
MKKTIMASSLAVALGVTGYAAGTGHQAHAAEVNVDQAHLVDLAHNHQDQLNAAPIKDGAYDIHFVKDGFQYNFTSNGTTWSWSYEAANGQTAGFSNVAGADYTTSYNQGSDVQSVSYNAQSSNSNVEAVSAPTYHNYSTSTTSSSVRLSNGNTAGATGSSAAQIMAQRTGVSASTWAAIIARESNGQVNAYNPSGASGLFQTMPGWGPTNTVDQQINAAVKAYKAQGLGAWGF

SEQ ID NO: 52 - protein, 202 aa, Artificial
  IsaA - Cloned peptide sequence
VNVDQAHLVDLAHNHQDQLNAAPIKDGAYDIHFVKDGFQYNFTSNGTTWSWSYEAANGQTAGFSNVAGADYTTSYNQGSDVQSVSYNAQSSNSNVEAVSAPTYHNYSTSTTSSSVRLSNGNTAGATGSSAAQIMAQRTGVSASTWAAIIARESNGQVNAYNPSGASGLFQTMPGWGPTNTVDQQINAAVKAYKAQGLGAWGF

SEQ ID NO: 53 - DNA, 930 bp, Staphylococcus aureus
atgactaactcttcgaaaagcttcactaaatttatggctgcttctgctgtttttactatgggatttttatcagtacctactgctggcgctgaacaaacaaatcaaattgcaaataaacctcaggctattcaatggcatacaaatttaacgaatgagcgattcactactatcgcacatcgtggcgcaagtggctatgcaccagagcatacgtttcaagcatatgataagagtcataatgagttaaaagcatcttatatcgaaattgatttacaacgtaccaaagatggacatttagttgctatgcatgatgaaactgttaaccgtacaacaaatggacacggtaaagttgaggattatacccttgatgaattaaaacagttagatgcaggaagttggtttaataaaaaatatccaaaatacgcaagagcaagttataaaaatgctaaagtacccactttagatgaaattttagaacgttatggcccgaatgcaaactattatattgaaacaaagtcacctgatgtatacccaggaatggaagaacaattattagcttcattgaaaaagcatcaccttttaaataacaataaattaaaaaatggacatgtaatgattcaatcattttctgacgaaagtttaaagaaaattcatcgtcaaaataagcatgtgccattagtaaaattagttgataaaggtgaactacaacaatttaacgaccaacgcttaaaagagatacgctcttatgcgattggattaggtcctgattatacagatttaactgaacaaaatacccatcatttaaaagacttaggatttatagtacatccttatacagtgaatgaaaaagctgatatgttacgattaaataaatatggcgttgatggtgtctttacaaatttcgctgataaatataaagaagtcattaagtag

SEQ ID NO: 54 - DNA, 837 bp, Artificial
  GlpQ - Cloned DNA Sequence
caaacaaatcaaattgcaaataaacctcaggctattcaatggcatacaaatttaacgaatgagcgattcactactatcgcacatcgtggcgcaagtggctatgcaccagagcatacgtttcaagcatatgataagagtcataatgagttaaaagcatcttatatcgaaattgatttacaacgtaccaaagatggacatttagttgctatgcatgatgaaactgttaaccgtacaacaaatggacacggtaaagttgaggattatacccttgatgaattaaaacagttagatgcaggaagttggtttaataaaaaatatccaaaatacgcaagagcaagttataaaaatgctaaagtacccactttagatgaaattttagaacgttatggcccgaatgcaaactattatattgaaacaaagtcacctgatgtatacccaggaatggaagaacaattattagcttcattgaaaaagcatcaccttttaaataacaataaattaaaaaatggacatgtaatgattcaatcattttctgacgaaagtttaaagaaaattcatcgtcaaaataagcatgtgccattagtaaaattagttgataaaggtgaactacaacaatttaacgaccaacgcttaaaagagatacgctcttatgcgattggattaggtcctgattatacagatttaactgaacaaaatacccatcatttaaaagacttaggatttatagtacatccttatacagtgaatgaaaaagctgatatgttacgattaaataaatatggcgttgatggtgtctttacaaatttcgctgataaatataaagaagtcattaagtag

SEQ ID NO: 55 - protein, 309 aa, Staphylococcus aureus
MTNSSKSFTKFMAASAVFTMGFLSVPTAGAEQTNQIANKPQAIQWHTNLTNERFTTIAHRGASGYAPEHTFQAYDKSHNELKASYIEIDLQRTKDGHLVAMHDETVNRTTNGHGKVEDYTLDELKQLDAGSWFNKKYPKYARASYKNAKVPTLDEILERYGPNANYYIETKSPDVYPGMEEQLLASLKKHHLLNNNKLKNGHVMIQSFSDESLKKIHRQNKHVPLVKLVDKGELQQFNDQRLKEIRSYAIGLGPDYTDLTEQNTHHLKDLGFIVHPYTVNEKADMLRLNKYGVDGVFTNFADKYKEVIK

SEQ ID NO: 56 - protein, 278 aa, Artificial
  GlpQ - Cloned peptide sequence
QTNQIANKPQAIQWHTNLTNERFTTIAHRGASGYAPEHTFQAYDKSHNELKASYIEIDLQRTKDGHLVAMHDETVNRTTNGHGKVEDYTLDELKQLDAGSWFNKKYPKYARASYKNAKVPTLDEILERYGPNANYYIETKSPDVYPGMEEQLLASLKKHHLLNNNKLKNGHVMIQSFSDESLKKIHRQNKHVPLVKLVDKGELQQFNDQRLKEIRSYAIGLGPDYTDLTEQNTHHLKDLGFIVHPYTVNEKADMLRLNKYGVDGVFTNFADKYKEVIK

SEQ ID NO: 57 - DNA, 1860 bp, Staphylococcus aureus
atgcctaaaaataaaattttaatttatttgctatcaactacgctcgtattacctactttagtttcacctaccgcttatgctgatacacctcaaaaagatactacagctaagacaacatctcatgattcaaaaaaatctaatgacgatgaaacttctaaggatactacaagtaaagatactgataaagcagacaacaataatacaagtaaccaagacaataacgacaaaaaatttaaaactatagacgacagcacttcagactctaacaatatcattgattttatttataagaatttaccacaaaccaatataaaccaattgctaaccaaaaataaatacgatgataattactcattaacaactttaatccaaaacttattcaatttaaattcggatatttctgattacgaacaacctcgtaatggcgaaaagtcaacaaatgattcgaataaaaacagtgacaatagcatcaaaaatgacactgatacgcaatcatctaaacaagataaagcagacaatcaaaaagcacctaaatcaaacaatacaaaaccaagtacatctaataagcaaccaaattcgccaaagccaacacaacctaatcaatcaaatagtcaaccagcaagtgacgataaagcaaatcaaaaatcttcatcgaaagataatcaatcaatgtcagattcggctttagactctattttggatcaatacagtgaagatgcaaagaaaacacaaaaagattatgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcattattcgaaacagatcctagtatatctaacaatgatgatagcggacaatttaacgttgttgactcaaaagatacacgtcaatttgtcaaatcaattgctaaagatgcacatcgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattagaagctgatggtaataaattgtatagtattaatgctggattccgaaaatatccaagcacgaaagaatcactaaaagattactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgcatgccagatttagataaatatgaacgttctatcaaggattatgatgattcatcagatgaattcaaacctttccgcgaggtatctgatagtatgccatatccacatggccaatgtacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaataa

SEQ ID NO: 58 - DNA, 1776 bp, Artificial
  Autolysin-like- Cloned DNA Sequence
acacctcaaaaagatactacagctaagacaacatctcatgattcaaaaaaatctaatgacgatgaaacttctaaggatactacaagtaaagatactgataaagcagacaacaataatacaagtaaccaagacaataacgacaaaaaatttaaaactatagacgacagcacttcagactctaacaatatcattgattttatttataagaatttaccacaaaccaatataaaccaattgctaaccaaaaataaatacgatgataattactcattaacaactttaatccaaaacttattcaatttaaattcggatatttctgattacgaacaacctcgtaatggcgaaaagtcaacaaatgattcgaataaaaacagtgacaatagcatcaaaaatgacactgatacgcaatcatctaaacaagataaagcagacaatcaaaaagcacctaaatcaaacaatacaaaaccaagtacatctaataagcaaccaaattcgccaaagccaacacaacctaatcaatcaaatagtcaaccagcaagtgacgataaagcaaatcaaaaatcttcatcgaaagataatcaatcaatgtcagattcggctttagactctattttggatcaatacagtgaagatgcaaagaaaacacaaaaagattatgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcattattcgaaacagatcctagtatatctaacaatgatgatagcggacaatttaacgttgttgactcaaaagatacacgtcaatttgtcaaatcaattgctaaagatgcacatcgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattagaagctgatggtaataaattgtatagtattaatgctggattccgaaaatatccaagcacgaaagaatcactaaaagattactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgcatgccagatttagataaatatgaacgttctatcaaggattatgatgattcatcagatgaattcaaacctttccgcgaggtatctgatagtatgccatatccacatggccaatgtacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaataa

SEQ ID NO: 59 - protein, 619 aa, Staphylococcus aureus
MPKNKILIYLLSTTLVLPTLVSPTAYADTPQKDTTAKTTSHDSKKSNDDETSKDTTSKDTDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLEADGNKLYSINAGFRKYPSTKESLKDYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQCTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 60 - protein, 591 aa, Artificial
  Autolysin-like- Cloned peptide sequence
TPQKDTTAKTTSHDSKKSNDDETSKDTTSKDTDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLEADGNKLYSINAGFRKYPSTKESLKDYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQCTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 61 - DNA, 960 bp, Staphylococcus aureus
atgaaaacacgtatagtcagctcagtaacaacaacactattgctaggttccatattaatgaatcctgtcgctaatgccgcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggttctatgaaagcagcagagaacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcattggacttcaacaaattggaaaggtaccaatactaaagataaatggacagatcgttcttcagaaagatataaaatcgattgggaaaaagaagaaatgacaaattaa

SEQ ID NO: 62 - DNA, 882 bp, Artificial
  Hla - Cloned DNA Sequence
gcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggttctatgaaagcagcagagaacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcattggacttcaacaaattggaaaggtaccaatactaaagataaatggacagatcgttcttcagaaagatataaaatcgattgggaaaaagaagaaatgacaaattaa

SEQ ID NO: 63 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKASKQQTNIDVIYERVRDDYQLHWTSTNWKGTNTKDKWTDRSSERYKIDWEKEEMTN

SEQ ID NO: 64 - protein, 293 aa, Artificial
  Hla - Cloned peptide sequence
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKASKQQTNIDVIYERVRDDYQLHWTSTNWKGTNTKDKWTDRSSERYKIDWEKEEMTN

SEQ ID NO: 65 - DNA, 1053 bp, Staphylococcus aureus
atgacaaaacattatttaaacagtaagtatcaatcagaacaacgttcatcagctatgaaaaagattacaatgggtacagcatctatcattttaggttcccttgtatacataggcgcagacagccaacaagtcaatgcggcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaataa

SEQ ID NO: 66 - DNA, 915 bp, Artificial
  IsdA - Cloned DNA Sequence
gcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaataa

SEQ ID NO: 67 - protein, 350 aa, Staphylococcus aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 68 - protein, 304 aa, Artificial
  IsdA- Cloned peptide sequence
ATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 69 - DNA, 1938 bp, Staphylococcus aureus
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 70 - DNA, 1818 bp, Artificial
  IsdB - Cloned DNA Sequence
gcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 71 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 72 - protein, 605 aa, Artificial
  IsdB - Cloned  peptide Sequence
AAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 73 - DNA, 4158 bp, Staphylococcus aureus
atgctaaacagagaaaataaaacggcaataacaagaaaaggcatggtatccaatcgattaaataaattttcgattagaaagtacacagtgggaacagcatcaattttagtaggtacaacattaatttttggtctggggaaccaagaagcaaaggctgcagaaagtactaataaagaattgaacgaagcgacaacttcagcaagtgataatcaatcgagtgataaagttgatatgcagcaactaaatcaagaagacaatactaaaaatgataatcaaaaagaaatggtatcatctcaaggtaatgaaacgacttcaaatgggaataaatcaatagaaaaagaaagtgtacaatctaccactggaaataaagttgaagtttcaactgccaaatcagatgagcaagcttcaccaaaatctacgaatgaagatttaaacactaaacaaactataagtaatcaagaagggttacaacctgatttgctagagaataaatcagtggtaaatgttcaaccaactaatgaggaaaacaaaaaggtagatgcgaaaactgaatcaactacattaaatgttaaaagtgatgctatcaagagtaatgctgaaactcttgttgataacaatagtaattcaaataatgaaaataatgcagatatcattttgccaaaaagtacagcacctaaaagtttgaatacaagaatgcgtatggcagcaatacaaccaaactcaacagattctaaaaatgttaatgatttaatcacatcaaatacaacattaactgtcgttgatgcagataatagcaaaacgattgtaccagcccaagattatttatcattaaaatcacaaattacagttgatgacaaagttaaatcaggtgattatttcacaattaaatactcagatacagtacaagtatatggattgaatccggaagatattaaaaatattggtgatattaaagatccaaataatggtgaaacaattgcgactgcaaaacatgatactgcaaataatttaattacatatacatttacagattatgttgatcgatttaattcagtaaaaatgggtattaattactcaatttatatggatgcagatacaattcctgttgacaagaaagatgttccttttagtgtaactattggaaatcaaattacaactacaacagcagatatcacttatccggcttataaagaagctgacaataattcaataggatcagcttttacagagacagtttctcatgtaggaaatgttgaagaccctggttactataaccaggtagtatatgttaatcctatggataaggatttaaaaggtgctaagttaaaagttgaagcgtaccatccgaaatatccaactaatattggtcaaattaatcaaaatgttacaaatataaaaatatatcgtgttcctgaaggatatacattgaataaaggatatgacgttaatactaatgatttggtagacgtaactgatgaatttaaaaataaaatgacgtatggatcaaatcaaagtgttaatcttgattttggtgatattacatcagcatatgttgtaatggttaatacaaaattccaatatacaaatagcgaaagcccaacacttgttcaaatggctactttatcttcaacaggtaataaatccgtttctactggcaatgctttaggatttactaataaccaaagtggcggagctggtcaagaagtatataaaattggtaactacgtatgggaagatactaataaaaacggtgttcaagaattaggagaaaaaggcgttggcaatgtaactgtaactgtatttgataataatacaaatacaaaagtaggagaagcagttactaaagaagatgggtcatacttgattccaaacttacctaatggagattaccgtgtagaattttcaaacttaccaaaaggttatgaagtaaccccttcaaaacaaggtaataacgaagaattagattcaaacggcttatcttcagttattacagttaatggcaaagataacttatctgcagacttaggtatttacaaacctaaatacaacttaggtgactatgtctgggaagatacaaataaaaatggtatccaagaccaagatgaaaaaggtatatctggcgtaacggtaacattaaaagatgaaaacggtaacgtgttaaaaacagttacaacagacgcagatggcaaatataaatttactgatttagataatggtaattataaagttgaatttactacaccagaaggctatacaccgactacagtaacatctggtagcgacattgaaaaagactctaatggtttaacaacaacaggtgttattaatggtgctgataacatgacattagatagtggattctacaaaacaccaaaatataatttaggtaattatgtatgggaagatacaaataaagatggtaagcaggattcaactgaaaaaggtatttcaggcgtaacagttacattgaaaaatgaaaacggtgaagttttacaaacaactaaaacagataaagatggtaaatatcaatttactggattagaaaatggaacttataaagttgaattcgaaacaccatcaggttacacaccaacacaagtaggttcaggaactgatgaaggtatagattcaaatggtacatcaacaacaggtgtcattaaagataaagataacgatactattgactctggtttctacaaaccgacttacaacttaggtgactatgtatgggaagatacaaataaaaacggtgttcaagataaagatgaaaagggtatttcaggtgtaacagttacgttaaaagatgaaaacgacaaagttttaaaaacagttacaacagatgaaaatggtaaatatcaattcactgatttaaacaatggaacttataaagttgaattcgagacaccatcaggttatacaccaacttcagtaacttctggaaatgatactgaaaaagattctaatggtttaacaacaacaggtgtcattaaagatgcagataacatgacattagacagtggtttctataaaacaccaaaatatagtttaggtgattatgtttggtacgacagtaataaagacggcaaacaagattcaactgaaaaaggtatcaaagatgttaaagttattttattaaatgaaaaaggcgaagtaattggaacaactaaaacagatgaaaatggtaaataccgctttgataatttagatagcggtaaatacaaagttatttttgaaaagcctactggcttaacacaaacaggtacaaatacaactgaagatgataaagatgccgatggtggcgaagttgatgtaacaattacggatcatgatgatttcacacttgataatggctactacgaagaagaaacatcagatagcgactcagattcggacagcgattcagactcagacagcgattcagactcagatagtgattcagattcagatagtgattcagattcagatagtgattcagattcagacagcgactcagactcagatagtgactcagactcagatagcgattcagattcagatagcgattcagactcagacagcgattcagattcagacagcgactcagactcagatagcgactcagattcggacagcgattcagactcagatagcgactcagactcagacagcgattcagactcagatagcgactcagactcagatagcgattcagattcagacagcgattcagattcagacagtgattcagattcagacagcgactcagattcagatagcgattcagactcagactcagatagcgattcagattcagacagcgactcagattcggacagcgactcagactcagacagtgattcagattcagatagcgactcagactcagatagcgactcagattcagacagcgattcagactcagatagtgactcagattcggacagcgattcagactcagatagcgactcagattcagacagtgattcagactcagatgcaggtaagcacacacctgttaaaccaatgagtactactaaagaccatcacaataaagcaaaagcattaccagaaactggtaatgaaaatagtggctcaaataacgcaacgttatttggcggattattcgcagcattaggatcattattgttattcggtcgtcgtaaaaaacaaaataaataa

SEQ ID NO: 74 - DNA, 2385 bp, Artificial
  SdrD - Partial Cloned DNA Sequence
ggcgttggcaatgtaactgtaactgtatttgataataatacaaatacaaaagtaggagaagcagttactaaagaagatgggtcatacttgattccaaacttacctaatggagattaccgtgtagaattttcaaacttaccaaaaggttatgaagtaaccccttcaaaacaaggtaataacgaagaattagattcaaacggcttatcttcagttattacagttaatggcaaagataacttatctgcagacttaggtatttacaaacctaaatacaacttaggtgactatgtctgggaagatacaaataaaaatggtatccaagaccaagatgaaaaaggtatatctggcgtaacggtaacattaaaagatgaaaacggtaacgtgttaaaaacagttacaacagacgcagatggcaaatataaatttactgatttagataatggtaattataaagttgaatttactacaccagaaggctatacaccgactacagtaacatctggtagcgacattgaaaaagactctaatggtttaacaacaacaggtgttattaatggtgctgataacatgacattagatagtggattctacaaaacaccaaaatataatttaggtaattatgtatgggaagatacaaataaagatggtaagcaggattcaactgaaaaaggtatttcaggcgtaacagttacattgaaaaatgaaaacggtgaagttttacaaacaactaaaacagataaagatggtaaatatcaatttactggattagaaaatggaacttataaagttgaattcgaaacaccatcaggttacacaccaacacaagtaggttcaggaactgatgaaggtatagattcaaatggtacatcaacaacaggtgtcattaaagataaagataacgatactattgactctggtttctacaaaccgacttacaacttaggtgactatgtatgggaagatacaaataaaaacggtgttcaagataaagatgaaaagggtatttcaggtgtaacagttacgttaaaagatgaaaacgacaaagttttaaaaacagttacaacagatgaaaatggtaaatatcaattcactgatttaaacaatggaacttataaagttgaattcgagacaccatcaggttatacaccaacttcagtaacttctggaaatgatactgaaaaagattctaatggtttaacaacaacaggtgtcattaaagatgcagataacatgacattagacagtggtttctataaaacaccaaaatatagtttaggtgattatgtttggtacgacagtaataaagacggcaaacaagattcaactgaaaaaggtatcaaagatgttaaagttattttattaaatgaaaaaggcgaagtaattggaacaactaaaacagatgaaaatggtaaataccgctttgataatttagatagcggtaaatacaaagttatttttgaaaagcctactggcttaacacaaacaggtacaaatacaactgaagatgataaagatgccgatggtggcgaagttgatgtaacaattacggatcatgatgatttcacacttgataatggctactacgaagaagaaacatcagatagcgactcagattcggacagcgattcagactcagacagcgattcagactcagatagtgattcagattcagatagtgattcagattcagatagtgattcagattcagacagcgactcagactcagatagtgactcagactcagatagcgattcagattcagatagcgattcagactcagacagcgattcagattcagacagcgactcagactcagatagcgactcagattcggacagcgattcagactcagatagcgactcagactcagacagcgattcagactcagatagcgactcagactcagatagcgattcagattcagacagcgattcagattcagacagtgattcagattcagacagcgactcagattcagatagcgattcagactcagactcagatagcgattcagattcagacagcgactcagattcggacagcgactcagactcagacagtgattcagattcagatagcgactcagactcagatagcgactcagattcagacagcgattcagactcagatagtgactcagattcggacagcgattcagactcagatagcgactcagattcagacagtgattcagactcagatgcaggtaagcacacacctgttaaaccaatgagtactactaaagaccatcacaataaagcaaaagcattaccagaaactggtaatgaaaatagtggctcaaataacgcaacgttatttggcggattattcgcagcattaggatcattattgttattcggtcgtcgtaaaaaacaaaataaataa

SEQ ID NO: 75 - DNA, 1524 bp, Artificial
  SdrD - Partial Cloned DNA Sequence
ggagaaaaaggcgttggcaatgtaactgtaactgtatttgataataatacaaatacaaaagtaggagaagcagttactaaagaagatgggtcatacttgattccaaacttacctaatggagattaccgtgtagaattttcaaacttaccaaaaggttatgaagtaaccccttcaaaacaaggtaataacgaagaattagattcaaacggcttatcttcagttattacagttaatggcaaagataacttatctgcagacttaggtatttacaaacctaaatacaacttaggtgactatgtctgggaagatacaaataaaaatggtatccaagaccaagatgaaaaaggtatatctggcgtaacggtaacattaaaagatgaaaacggtaacgtgttaaaaacagttacaacagacgcagatggcaaatataaatttactgatttagataatggtaattataaagttgaatttactacaccagaaggctatacaccgactacagtaacatctggtagcgacattgaaaaagactctaatggtttaacaacaacaggtgttattaatggtgctgataacatgacattagatagtggattctacaaaacaccaaaatataatttaggtaattatgtatgggaagatacaaataaagatggtaagcaggattcaactgaaaaaggtatttcaggcgtaacagttacattgaaaaatgaaaacggtgaagttttacaaacaactaaaacagataaagatggtaaatatcaatttactggattagaaaatggaacttataaagttgaattcgaaacaccatcaggttacacaccaacacaagtaggttcaggaactgatgaaggtatagattcaaatggtacatcaacaacaggtgtcattaaagataaagataacgatactattgactctggtttctacaaaccgacttacaacttaggtgactatgtatgggaagatacaaataaaaacggtgttcaagataaagatgaaaagggtatttcaggtgtaacagttacgttaaaagatgaaaacgacaaagttttaaaaacagttacaacagatgaaaatggtaaatatcaattcactgatttaaacaatggaacttataaagttgaattcgagacaccatcaggttatacaccaacttcagtaacttctggaaatgatactgaaaaagattctaatggtttaacaacaacaggtgtcattaaagatgcagataacatgacattagacagtggtttctataaaacaccaaaatatagtttaggtgattatgtttggtacgacagtaataaagacggcaaacaagattcaactgaaaaaggtatcaaagatgttaaagttattttattaaatgaaaaaggcgaagtaattggaacaactaaaacagatgaaaatggtaaataccgctttgataatttagatagcggtaaatacaaagttatttttgaaaagcctactggcttaacacaaacaggtacaaatacaactgaagatgataaataa

SEQ ID NO: 76 - protein, 1385 aa, Staphylococcus aureus
MLNRENKTAITRKGMVSNRLNKFSIRKYTVGTASILVGTTLIFGLGNQEAKAAESTNKELNEATTSASDNQSSDKVDMQQLNQEDNTKNDNQKEMVSSQGNETTSNGNKSIEKESVQSTTGNKVEVSTAKSDEQASPKSTNEDLNTKQTISNQEGLQPDLLENKSVVNVQPTNEENKKVDAKTESTTLNVKSDAIKSNAETLVDNNSNSNNENNADIILPKSTAPKSLNTRMRMAAIQPNSTDSKNVNDLITSNTTLTVVDADNSKTIVPAQDYLSLKSQITVDDKVKSGDYFTIKYSDTVQVYGLNPEDIKNIGDIKDPNNGETIATAKHDTANNLITYTFTDYVDRFNSVKMGINYSIYMDADTIPVDKKDVPFSVTIGNQITTTTADITYPAYKEADNNSIGSAFTETVSHVGNVEDPGYYNQVVYVNPMDKDLKGAKLKVEAYHPKYPTNIGQINQNVTNIKIYRVPEGYTLNKGYDVNTNDLVDVTDEFKNKMTYGSNQSVNLDFGDITSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVILLNEKGEVIGTTKTDENGKYRFDNLDSGKYKVIFEKPTGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGNENSGSNNATLFGGLFAALGSLLLFGRRKKQNK

SEQ ID NO: 77 - protein, 794 aa, Artificial
  SdrD - Partial cloned peptide sequence
GVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVILLNEKGEVIGTTKTDENGKYRFDNLDSGKYKVIFEKPTGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGNENSGSNNATLFGGLFAALGSLLLFGRRKKQNK

SEQ ID NO: 78 - protein, 507 aa, Artificial
  SdrD - Partial cloned peptide sequence
GEKGVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVILLNEKGEVIGTTKTDENGKYRFDNLDSGKYKVIFEKPTGLTQTGTNTTEDDK

SEQ ID NO: 79 - DNA, 2808 bp, Staphylococcus aureus
atgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagaatacagatgcaagtaataaagatgtagttagtcaagcggttaatccaagtacgcctagaatgagagcatttagtttagcggcagtagctgcagatgcaccggcagctggcacagatattacgaatcagttgacagatgtgaaagttactattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgttgataataaagaaaatgtaacagctaatattactatgccagcttatattgaccctgaaaatgttacaaagacaggtaatgtgacattgacaactggcataggaaccaatactgctagtaagacagtattaatcgactatgagaaatatggacaattccataatttatcaattaaaggtacgattgatcaaatcgataaaacaaataatacgtatcgccaaacaatttatgtcaatccaagcggagataacgttgtgttacctgccttaacaggtaatttaattcctaatacaaagagtaatgcgttaatagatgcaaaaaacactgatattaaagtttatagagtcgataatgctaatgatttatctgaaagttattatgtgaatcctagcgattttgaagatgtaactaatcaagttagaatttcatttccaaatgctaatcaatacaaagtagaatttcctacggacgatgaccaaattacaacaccgtatattgtagttgttaatggccatattgatcctgctagtacaggtgatttagcactacgttcgacattttatggttatgattctaattttatatggagatctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattctggcagtgattctacatcagatagtggttcagattcagcgagtgattcagattcagcaagtgattcagactcagcgagtgattcagattcagcaagtgattcagattcagcaagtgattcagattcagcaagtgattcagactcagcaagtgattcagattcagcaagtgattcagattcagcaagcgattcagattcagcgagcgattcagattcagcgagcgattcagattcagcgagtgattccgactcagcgagcgattcagactcagatagtgactcagattccgatagcgattccgactcagatagcgactcagattcagacagcgattctgactcagacagcgattctgactcagacagtgactcagattccgatagcgattctgactcagacagtgactcagattccgatagcgattcagattcagacagtgattcagactcagatagcgattcagattccgacagtgactcagactcagacagcgattcagattccgatagcgattcagattccgacagtgactcagattccgatagtgactcggattcagcgagtgattcagattcagatagcgattcagaatcagatagtgactcagactcagacagtgattcagattcagatagtgactcagactcagacagcgattcagaatcagatagtgactccgattcagacagcgattcagaatcagatagtgactccgattcagatagcgattcggattcagcgagtgattcagactcaggtagtgactccgattcatcaagtgattcagattccgattcaacgagtgacacaggatcagacaacgactcagacagtgattcaaatagcgattccgagtcaggttctaacaataatgtagttccgcctaattcacctaaaaatggtactaatgcttctaataaaaatgaggctaaagatagtaaagaaccattaccagatacaggttctgaagatgaagcgaatacgtcactaatttggggattattagcatcattaggttcattactacttttcagaagaaaaaaagaaaataaagataagaaataa

SEQ ID NO: 80 - DNA, 1638 bp, Artificial
  ClfA - Partial Cloned DNA Sequence
gaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagaatacagatgcaagtaataaagatgtagttagtcaagcggttaatccaagtacgcctagaatgagagcatttagtttagcggcagtagctgcagatgcaccggcagctggcacagatattacgaatcagttgacagatgtgaaagttactattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgttgataataaagaaaatgtaacagctaatattactatgccagcttatattgaccctgaaaatgttacaaagacaggtaatgtgacattgacaactggcataggaaccaatactgctagtaagacagtattaatcgactatgagaaatatggacaattccataatttatcaattaaaggtacgattgatcaaatcgataaaacaaataatacgtatcgccaaacaatttatgtcaatccaagcggagataacgttgtgttacctgccttaacaggtaatttaattcctaatacaaagagtaatgcgttaatagatgcaaaaaacactgatattaaagtttatagagtcgataatgctaatgatttatctgaaagttattatgtgaatcctagcgattttgaagatgtaactaatcaagttagaatttcatttccaaatgctaatcaatacaaagtagaatttcctacggacgatgaccaaattacaacaccgtatattgtagttgttaatggccatattgatcctgctagtacaggtgatttagcactacgttcgacattttatggttatgattctaattttatatggagatctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattctggcagtgattcttaa

SEQ ID NO: 81 - DNA, 1353 bp, Artificial
  ClfA - Partial Cloned DNA Sequence
ccgtatattgtagttgttaatggccatattgatcctgctagtacaggtgatttagcactacgttcgacattttatggttatgattctaattttatatggagatctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattctggcagtgattctacatcagatagtggttcagattcagcgagtgattcagattcagcaagtgattcagactcagcgagtgattcagattcagcaagtgattcagattcagcaagtgattcagattcagcaagtgattcagactcagcaagtgattcagattcagcaagtgattcagattcagcaagcgattcagattcagcgagcgattcagattcagcgagcgattcagattcagcgagtgattccgactcagcgagcgattcagactcagatagtgactcagattccgatagcgattccgactcagatagcgactcagattcagacagcgattctgactcagacagcgattctgactcagacagtgactcagattccgatagcgattctgactcagacagtgactcagattccgatagcgattcagattcagacagtgattcagactcagatagcgattcagattccgacagtgactcagactcagacagcgattcagattccgatagcgattcagattccgacagtgactcagattccgatagtgactcggattcagcgagtgattcagattcagatagcgattcagaatcagatagtgactcagactcagacagtgattcagattcagatagtgactcagactcagacagcgattcagaatcagatagtgactccgattcagacagcgattcagaatcagatagtgactccgattcagatagcgattcggattcagcgagtgattcagactcaggtagtgactccgattcatcaagtgattcagattccgattcaacgagtgacacaggatcagacaacgactcagacagtgattcaaatagcgattccgagtcaggttctaacaataatgtagttccgcctaattcacctaaaaatggtactaatgcttctaataaaaatgaggctaaagatagtaaagaaccattaccagatacaggttctgaagatgaagcgaatacgtcactaatttggggattattagcatcattaggttcattactacttttcagaagaaaaaaagaaaataaagataagaaataa

SEQ ID NO: 82 - protein, 935 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQNTDASNKDVVSQAVNPSTPRMRAFSLAAVAADAPAAGTDITNQLTDVKVTIDSGTTVYPHQAGYVKLNYGFSVPNSAVKGDTFKITVPKELNLNGVTSTAKVPPIMAGDQVLANGVIDSDGNVIYTFTDYVDNKENVTANITMPAYIDPENVTKTGNVTLTTGIGTNTASKTVLIDYEKYGQFHNLSIKGTIDQIDKTNNTYRQTIYVNPSGDNVVLPALTGNLIPNTKSNALIDAKNTDIKVYRVDNANDLSESYYVNPSDFEDVTNQVRISFPNANQYKVEFPTDDDQITTPYIVVVNGHIDPASTGDLALRSTFYGYDSNFIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSASDSDSASDSDSASDSDSASDSDSASDSDSASDSDSASDSDSASDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSESDSDSDSDSDSDSDSDSDSDSDSESDSDSDSDSDSESDSDSDSDSDSDSASDSDSGSDSDSSSDSDSDSTSDTGSDNDSDSDSNSDSESGSNNNVVPPNSPKNGTNASNKNEAKDSKEPLPDTGSEDEANTSLIWGLLASLGSLLLFRRKKENKDKK

SEQ ID NO: 83 - protein, 545 aa, Artificial
  ClfA -Partial cloned peptide sequence
ENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQNTDASNKDVVSQAVNPSTPRMRAFSLAAVAADAPAAGTDITNQLTDVKVTIDSGTTVYPHQAGYVKLNYGFSVPNSAVKGDTFKITVPKELNLNGVTSTAKVPPIMAGDQVLANGVIDSDGNVIYTFTDYVDNKENVTANITMPAYIDPENVTKTGNVTLTTGIGTNTASKTVLIDYEKYGQFHNLSIKGTIDQIDKTNNTYRQTIYVNPSGDNVVLPALTGNLIPNTKSNALIDAKNTDIKVYRVDNANDLSESYYVNPSDFEDVTNQVRISFPNANQYKVEFPTDDDQITTPYIVVVNGHIDPASTGDLALRSTFYGYDSNFIWRSMSWDNEVAFNNGSGSGDGIDKPVVPEQPDEPGEIEPIPEDSDSDPGSDSGSDSNSDSGSDSGSDS

SEQ ID NO: 84 - protein, 450 aa, Artificial
  ClfA -Partial cloned peptide sequence
PYIVVVNGHIDPASTGDLALRSTFYGYDSNFIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSASDSDSASDSDSASDSDSASDSDSASDSDSASDSDSASDSDSASDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSESDSDSDSDSDSDSDSDSDSDSDSESDSDSDSDSDSESDSDSDSDSDSDSASDSDSGSDSDSSSDSDSDSTSDTGSDNDSDSDSNSDSESGSNNNVVPPNSPKNGTNASNKNEAKDSKEPLPDTGSEDEANTSLIWGLLASLGSLLLFRRKKENKDKK

SEQ ID NO: 85 - DNA, 717 bp, Artificial
  GFP - Cloned DNA sequence
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa

SEQ ID NO: 86 - protein, 238 aa, Artificial
  GFP - Cloned peptide sequence
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

(19) United States
(12) Patent Application Publication
(10) Pub. No.: US 2024/0042002 A1
(43) Pub. Date: Feb. 8, 2024

(54) Compositions and methods for the prevention of S. aureus infection

(71) Applicants: ANTAGONIS, MONTIGNY-LE-BRETONNEUX (FR); UNIVERSITE DE VERSAILLES-ST QUENTIN EN YVELINES, VERSAILLES CEDEX (FR); ASSISTANCE PUBLIQUE - HOPITAUX DE PARIS, PARIS (FR)

(72) Inventors: Cassie RIBEIRO, BUC (FR); Martin ROTTMAN, LA CELLE SAINT-CLOUD (FR); Jean-Louis GAILLARD, ISSY LES MOULINEAUX (FR)

(21) Appl. No.: 17/789,451

(22) PCT Filed: Dec. 31, 2020
(86) PCT No.: PCT/EP2020/088082

§ 371 Date: Jun. 27, 2022

(30) Foreign Application Priority Data
Dec. 31, 2019 (EP) 19306797.2

(51) Int. Cl. A61K 39/085; A61K 39/40; A61P 31/04; G01N 33/569; C12Q 1/18

(52) U.S. Cl. CPC A61K 39/085; A61K 39/40; A61P 31/04; G01N 33/56938; C12Q 1/18; G01N 2333/31; G01N 2469/10

(57) ABSTRACT

The present invention relates to an immunogenic composition comprising at least one Staphylococcus aureus antigen, wherein said antigen is a polypeptide having at least 80% identity with the SdrH-like polypeptide of SEQ ID NO: 8, Nuc of SEQ ID

NO: 4, or LukG of SEQ ID NO: 12, an immunotherapeutic composition comprising a polyclonal antibody which selectively binds to at least one of said antigens, and an in vitro method of identifying an antigen conferring protection against disease caused by S. aureus in a subject.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Dec. 9, 2022, is named 1778945_ST25.txt and is 246,662 bytes in size.

### INTRODUCTION

The present invention relates to immunogenic compositions comprising Staphylococcus aureus antigens. The present invention further relates to immunogenic compositions for use in conferring protection against disease caused by S. aureus in a subject.

S. aureus is a major cause of infection in humans, and is responsible for a wide range of pathologies including skin and soft tissue infections, osteomyelitis, endocarditis, and sepsis. In particular, S. aureus is responsible for a high proportion of infections associated with foreign devices (e.g., catheters) and implants (e.g., prosthetics), due to its ability to form a biofilm on the surfaces of these materials. These infections are particularly problematic as they may be chronic or systemic, in some cases causing prosthetic joint infection, implant failure, or even death. As an example, retrospective analysis of S. aureus infections in a large hemodialysis center found that the rate of S. aureus infection in hemodialysis patients was nearly 18% and was associated with a 10% mortality rate, with the large majority of infections being associated with vascular catheters (Fitzgerald et al., 2011).

While S. aureus infections are typically treated with antibiotic therapies, the emergence of antibiotic resistant S. aureus bacteria, including methicillin-resistant (MRSA) and vancomycin-resistant (VRSA) strains have complicated the use of conventional antibiotics. Furthermore, while vancomycin is currently the gold standard for treatment of MRSA bacteremia and endocarditis, this antibiotic not ideal due to poor tissue penetration, undesirable side effects, and slow bactericidal activity (Gould, 2008). In the case of orthopedic implants, revision surgery is most often required (Darouiche, 2004). However, this strategy is costly and invasive, and is often more technically difficult than the initial implant surgery, requires more extensive surgery and is associated with lower quality of life outcomes in subjects.

The development of effective vaccines preventing S. aureus infection represents a promising alternative to current treatment methods, with various vaccines against S. aureus currently under evaluation in phase I, II, or III clinical trials, though no successful phase III trial has yet been completed. To date, vaccine development has focused mainly on the S. aureus secreted alpha toxin (Hla) and/or on the capsular polysaccharide. While a vaccine targeting alpha toxin has been shown to have a protective effect against infections in which the toxin is responsible for the majority of the pathogenic effect (e.g., pneumonia, as described in Bubeck and Schneewind, 2008), it is insufficient for preventing sub-lethal infections (Adlam et al., 1977). Furthermore, given the variability of capsular polysaccharides, and the fact that a large number of methicillin-resistant S. aureus strains are unencapsulated, the use of capsular polysaccharide alone is of limited interest. Various S. aureus proteins taken alone or in combination are also under evaluation, including the IsdB protein, which was shown to induce antibodies in subjects having S. aureus infection, although this was insufficient to provide protection against future infection (Zorman et al., 2013). Clinical trials have further shown that vaccination with IsdB has no effect when compared to placebo recipients, and in some cases is even deleterious (McNeely et al., 2014).

As current strategies are unsatisfactory, there remains a need for improved immunogenic compositions comprising S. aureus antigens or polyclonal antibodies raised against said antigens. In particular, there is a need for novel immunogenic and immunotherapeutic compositions that are able to prevent and/or treat S. aureus infection, for example comprising antigens which are able to induce protective antibodies against S. aureus infection or such antibodies themselves. There also remains a need for novel methods of identifying antigens conferring protection against disease caused by S. aureus in a subject. In particular, in view of the high cost and duration of clinical trials there exists a need for an improved assay that may be used as a correlate of protection for assessing vaccine responses.

The present invention fulfils these and other needs by providing an immunogenic composition, an immunotherapeutic composition, and an in vitro method of identifying an antigen conferring protection against disease caused by S. aureus in a subject.

In particular, the present invention provides an immunogenic composition comprising at least one Staphylococcus aureus antigen, wherein said antigen is a polypeptide having at least 80% identity with the SdrH-like polypeptide of SEQ ID NO: 8, Nuc of SEQ ID NO: 4, or LukG of SEQ ID NO: 12.

According to a particular aspect, the immunogenic composition comprises an antigen having at least 80% identity with the SdrH-like polypeptide of SEQ ID NO: 8 and an antigen having at least 80% identity with LukG of SEQ ID NO: 12.

The one or more antigens comprised in the immunogenic composition of the invention advantageously provide unexpected, improved immunogenic properties (e.g., level, quality and/or scope of the immunogenic response) as compared to existing antigens, such as IsdB.

Preferably, the immunogenic composition comprises the S. aureus antigens in the form of separate polypeptides or in the form of one or more fusion polypeptides or both in the form of separate polypeptide(s) and fusion polypeptide(s).

Preferably, the immunogenic composition further comprises a pharmaceutically acceptable excipient.

Preferably, the immunogenic composition is for use as a vaccine conferring protection against disease caused by S. aureus in a subject

The present invention further relates to an immunotherapeutic composition comprising a polyclonal antibody which selectively binds to at least one antigen as defined herein, wherein said antibody promotes uptake and killing of S. aureus by phagocytes.

Preferably, the immunotherapeutic composition further comprises a pharmaceutically acceptable excipient.

Preferably, the immunotherapeutic composition is for use as a passive immunotherapy conferring protection against disease caused by S. aureus in a subject.

Preferably, said S. aureus is a methicillin-resistant S. aureus (MRSA) or a methicillin-susceptible S. aureus (MSSA).

Preferably, said subject has an osteoarticular device, preferably an osteoarticular implant, more preferably a total joint replacement prosthesis.

Preferably, said immunogenic or immunotherapeutic composition provided herein is for use in association with one or more antibiotics effective against a S. aureus infection.

The present invention further relates to an in vitro method of identifying an antigen conferring protection against disease caused by S. aureus in a subject comprising:
a) incubating a solution comprising S. aureus with a solution comprising antibodies raised against an S. aureus antigen, preferably for one hour at 35° C., thereby obtaining a mixed suspension,
b) contacting macrophages with the mixed suspension of step a),
c) removing the mixed suspension from macrophages and adding fresh medium supplemented with antibiotics to kill extracellular S. aureus bacteria, and
d) assessing internalization and killing of S. aureus bacteria by said macrophages, wherein said antigen is considered to confer protection against disease caused by S. aureus when said antigen induces both increased internalization and killing of S. aureus while preserving the viability of macrophages.

In contrast to previous methods, which notably use polymorphonuclear neutrophils, the inventors have developed a novel OPA assay for identifying target vaccine antigens capable of generating antibodies promoting both uptake and killing of S. aureus. Indeed, polymorphonuclear neutrophils show very strong bactericidal activity (“killing”), which notably makes it impossible to evaluate whether or not eventual “facilitating” antibodies (i.e., which promote bacterial uptake but which then result in intracellular bacterial growth rather than killing), are generated. Advantageously, macrophages have a much lower bactericidal (“killing”) activity than polymorphonuclear neutrophils, due to their lower levels of synthesis of reactive oxygen species and antimicrobial peptides. Furthermore, in the specific context of osteoarticular prosthetic infections, the development of a macrophage-based assay is particularly advantageous, as, in the physiopathology of infection, circulating blood-borne S. aureus must be cleared from the bloodstream by macrophages present in the spleen and/or lungs rather than by polymorphonuclear neutrophils, thereby reducing the duration of bacteremia and the probability of establishing a prosthetic infection.

Preferably, said macrophages are an immortalized macrophage cell line, preferably the J774.2 cell line.

Preferably, the killing of S. aureus bacteria in step d) is assessed by comparing the quantity of bacteria internalized in macrophages 3 hours after step c) with the quantity of bacteria internalized in macrophages 6 hours after step c).

### DESCRIPTION OF THE INVENTION

Before describing the invention in further detail, it should be noted that the terms “a” and “an” as used herein are used in the sense that they mean “at least one”, “at least a first”, “one or more” or “a plurality” of the referenced compounds or steps, unless the context dictates otherwise. The term “and/or” as used herein includes the meaning of “and”, “or” and “all or any other combination of the elements connected by said term”. The term “comprising”, “having”, “including”, or “containing” (and any form of said terms, such as e.g., “contains” or “contain”) are open-ended and do not exclude additional, unrecited elements or method steps. In contrast, the term “consisting of” as used herein excludes any other components (beyond trace levels) or steps.

As indicated above, according to a first aspect, the present invention relates to an immunogenic composition comprising at least one Staphylococcus aureus antigen, wherein said antigen is a polypeptide having at least 80% identity with the SdrH-like polypeptide of SEQ ID NO: 8, Nuc of SEQ ID NO: 4, or LukG of SEQ ID NO: 12.

The term “immunogenic” as used herein refers to the ability of the composition to induce or stimulate a measurable B cell-mediated immune response in a subject into which the component qualified as immunogenic has been introduced. For example, the composition of the invention is immunogenic in the sense that it is capable of inducing or stimulating an immune response in a subject which can be innate and/or specific (i.e., against at least one S. aureus polypeptide comprised in said immunogenic composition), humoral and/or cellular (e.g., production of antibodies and/or cytokines and/or the activation of cytotoxic T cells, B, T lymphocytes, antigen presenting cells, helper T cells, dendritic cells, NK cells, etc). The immunogenic composition usually results in a protective response in the administered subject. Specifically, the composition of the invention is immunogenic in that it induces antibodies recognizing at least one S. aureus polypeptide and increases both the uptake and the killing of S. aureus by phagocytes. However, said composition may also induce one or more additional immune responses.

Specifically, the inventors have surprisingly shown here that each of the SdrH-like polypeptide, Nuc, and LukG antigens is able to induce antibodies increasing both the uptake and the killing of S. aureus by phagocytes. The generation of antibodies having such activity, performed for the first time here in a macrophage-based model, is indicative that the antigens described herein induce protection against S. aureus infection when present in an immunogenic composition. Indeed, the results obtained here, with the antigens of the invention, are in notable contrast with those obtained with IsdB, previously shown to have deleterious effects (as S. aureus infection may be favored), confirming the pertinence of this macrophage-based model in evaluating antigens. In vivo results obtained in an animal model further show that these antigens are able to reduce S. aureus bacterial growth in the kidneys to a larger extent than that observed with adjuvant alone and/or with a control antigen such as staphylokinase, further confirming their ability to induce protection against S. aureus infection. Thus, according to a particular aspect, the immunogenic composition comprises at least one Staphylococcus aureus antigen inducing antibodies against said antigen increasing both the uptake and the killing of S. aureus upon phagocytosis of the bacteria, wherein said antigen is a polypeptide having at least 80% identity with the SdrH-like polypeptide of SEQ ID NO: 8, Nuc of SEQ ID NO: 4, or LukG of SEQ ID NO: 12.

As used herein, the term “S. aureus antigen” refers to a polypeptide present in or obtained from a S. aureus species or a fragment thereof (e.g., an epitope) capable of being bound by an antibody, wherein said antigen is selected from an “SdrH-like” polypeptide, Nuc, and LukG, and combinations of one or more thereof. Typically, such an antigen contains one or more B epitope(s). In the context of the invention, this term encompasses native S. aureus antigens (e.g., a full-length antigen) or modified versions (e.g., fragments or variants) thereof. A “native” S. aureus antigen can notably be found, isolated, obtained from a source of S. aureus in nature. Such sources include biological samples (e.g., blood, plasma, sera, saliva, sputum, tissue sections, biopsy specimens, etc.) collected from a subject that has been infected with or exposed to S. aureus, cultured cells, as well as recombinant materials available in depositary institutions (e.g., ATCC or TB institutions), libraries or described in the literature (e.g., S. aureus isolates, S. aureus genomes, etc.).

The “SdrH-like” antigen or polypeptide is a cell wall-anchored serine-aspartate repeat family protein containing the host attachment domain MSCRAMM (microbial surface components recognizing adhesive matrix molecules). The “SdrH-like” polypeptide may comprise the sequence of SEQ ID NO: 7 or 8, which may be encoded by the nucleotide sequence of SEQ ID NO: 5 or 6, respectively. In the context of the present invention, the “SdrH-like” polypeptide preferably has the sequence of SEQ ID NO: 8.

The “Nuc” antigen (also known as micrococcal nuclease or thermonuclease) is an extracellular nuclease. After cleavage by a signal peptidase at the cell membrane, Nuc may be processed into two active forms: NucA or NucB. Nuc may notably comprise the sequence of SEQ ID NO: 3 or 4, which may be encoded by the nucleotide sequence of SEQ ID NO: 1 or 2. In the context of the present invention, Nuc preferably has the sequence of SEQ ID NO: 4.

The “LukG” antigen (also known as LukA) forms a heterodimer with “LukH” (also known as LukB). This heterodimer, LukGH, is a pore-forming leucocidin that at least partially mediates killing of immune cells, such as human monocytes, macrophages, and polymorphonuclear cells by S. aureus. LukG may comprise the sequence of SEQ ID NO: 11 or 12, which may be encoded by the nucleotide sequence of SEQ ID NO: 9 or 10, respectively. In the context of the present invention, LukG preferably has the sequence of SEQ ID NO: 12.

While LukG forms a heterodimer with LukH, preferably said immunogenic composition comprises LukG in the absence of LukH. Indeed, the inventors have surprisingly found that antibodies increasing the uptake and the killing of S. aureus by phagocytes may be induced by LukG when taken alone (i.e., in the absence of LukH). This is in notable contrast to previous studies which suggest that the LukGH heterodimer must be used to generate antibodies. In particular, Badarau et al. 2016 found that monoclonal antibodies raised against either LukG or LukH alone had very little or even no ability to neutralize the LukGH toxin. Thus, according to a preferred aspect, while the immunogenic composition may comprise both LukG and LukH it preferably comprises LukG in the absence of LukH.

The skilled person will understand that, as a result of the degeneracy of the genetic code, there are many nucleotide sequences that may encode a polypeptide as described herein. In particular, codon usage within a given nucleotide sequence may be adapted for optimized expression of the corresponding polypeptide in an organism other than S. aureus (e.g., E. coli).

A modified S. aureus antigen (e.g., a variant) typically differs from a polypeptide specifically disclosed herein or a native polypeptide at one or more position(s), for example via one or more amino acid substitutions, insertions, additions and/or deletions, non-natural arrangements, and any combination thereof. Amino acid substitutions may be equivalent or not. Preferably, the substitution is made with an “equivalent” amino acid, i.e., any amino acid whose structure is similar to that of the original amino acid and therefore unlikely to change the biological activity of the antigen. Examples of such substitutions are presented in Table 1 below:

TABLE 1
Substitutions with equivalent amino acids

| Original amino acid | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

When several modifications are contemplated, they may concern consecutive and/or non-consecutive residues. Modification(s) may be generated by a number of ways known to the skilled person, such as site-directed mutagenesis, PCR mutagenesis, DNA shuffling and by synthetic techniques (e.g., resulting in a synthetic nucleic acid molecule encoding the desired polypeptide variant).

Regardless of the origin of the S. aureus antigen (e.g., native or modified), the antigen comprised in the immunogenic composition of the invention retains one or more immunogenic portions of the corresponding native antigen, more preferably B epitope(s). Methods to identify the appropriate immunogenic portion of an antigen are well-known in the art.

The term “polypeptide” as used herein refers to a polymer of amino acid residues which comprises at least 10 or more amino acids, preferably at least 20 or more amino acids, bonded via covalent peptide bonds. The polypeptide may be linear, branched or cyclic and may comprise naturally occurring and/or amino acid analogs. It may be chemically modified (e.g., being glycosylated, lipidated, acetylated, cleaved, cross-linked by disulfide bridges and/or phosphorylated). It may comprise additional elements such as a tag (e.g., his, myc, Flag, etc.) and/or a targeting peptide (e.g., signal peptide, trans-membrane domain, etc.). Preferably, the at least one polypeptide comprised in the immunogenic composition of the present invention does not comprise a signal peptide. Preferably, the at least one polypeptide comprised in the immunogenic composition of the invention does not comprise a tag. It will be understood that the term “polypeptide” encompasses proteins (usually employed for polypeptides comprising 50 or more amino acid residues), oligopeptides, and peptides (usually employed for polypeptides comprising less than 50 amino acid residues). Each polypeptide may thus be characterized by specific amino acids and be encoded by specific nucleic acid sequences, such as those provided herein.

Thus, a polypeptide “comprises” an amino acid sequence when the amino acid sequence is a part of the final amino acid sequence of the polypeptide. Such a polypeptide may in some cases have up to several hundred additional amino acids residues (e.g., tag peptides, targeting peptides, etc.). A polypeptide “consists of” an amino acid sequence when the polypeptide does not contain any amino acids other than that of the recited amino acid sequence.

The term “percent (%) identity” refers to an amino acid to amino acid or nucleotide to nucleotide correspondence between two polypeptide or nucleic acid molecules. The percentage of identity between two molecules is a function of the number of identical positions shared by the sequences, taking into account the number of gaps which must be introduced for optimal alignment and the length of each gap. The percent identities referred to in the context of the present invention are determined after optimal alignment of the sequences to be compared, which may therefore comprise one or more insertions, deletions, truncations and/or substitutions. This percent identity may be calculated by any sequence analysis method well-known to the person skilled in the art. In particular, the percent identity may be determined after global alignment of the sequences to be compared of the sequences taken in their entirety over their entire length. In addition to manual comparison, it is possible to determine global alignment using the algorithm of Needleman and Wunsch (1970).

For nucleotide sequences, the sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4).

For amino acid sequences, the sequence comparison may be performed using any software well-known to a person skilled in the art, such as the Needle software. The parameters used may notably be the following: “Gap open” equal to 10.0, “Gap extend” equal to 0.5, and the BLOSUM62 matrix.

Preferably, the percent identify as defined in the context of the present invention is determined via the global alignment of sequences compared over their entire length.

The present invention encompasses polypeptide sequences having substantial sequence identity to the polypeptides disclosed herein, preferably comprising at least 50% sequence identity, preferably at least 60%, 70%, 75%, 80%, 85%, 86%, 87%, 88%, 89%, 90%, 91%, 92%, 93%, 94%, 95%, 96%, 97%, 98%, or 99% or higher, sequence identity with a polypeptide sequence provided herein using the methods described above. According to a preferred embodiment, the polypeptide has at least 80% identity with the SdrH-like polypeptide, Nuc, or LukG of S. aureus subsp. aureus Mu50 (Accession no. BA000017.4). Preferably, the polypeptide has at least 80% identity with the SdrH-like polypeptide of SEQ ID NO: 8, Nuc of SEQ ID NO: 4, or LukG of SEQ ID NO: 12, even more preferably at least 85%, 86%, 87%, 88%, 89%, 90%, 91%, 92%, 93%, 94%, 95%, 96%, 97%, 98%, 99% or higher identity. Preferably, the polypeptide has 100% identity with the SdrH-like polypeptide of SEQ ID NO: 8, Nuc of SEQ ID NO: 4, or LukG of SEQ ID NO: 12.

The immunogenic composition provided herein may comprise any combination of the polypeptides provided herein. As a non-limiting example, the composition may comprise a polypeptide having at least 80% identity with Nuc of SEQ ID NO: 4 and a polypeptide having at least 80% identity with LukG of SEQ ID NO: 12. Alternatively, the composition may comprise a polypeptide having at least 80% identity with Nuc of SEQ ID NO: 4 and a polypeptide having at least 80% identity with the SdrH-like polypeptide of SEQ ID NO: 8. Alternatively, the composition may comprise a polypeptide having at least 80% identity with the SdrH-like polypeptide of SEQ ID NO: 8 and an antigen having at least 80% identity with LukG of SEQ ID NO: 12. Alternatively, the composition may comprise a polypeptide having at least 80% identity with Nuc of SEQ ID NO: 4, a polypeptide having at least 80% identity with LukG of SEQ ID NO: 12, and a polypeptide having at least 80% identity with the SdrH-like polypeptide of SEQ ID NO: 8. Preferably, the immunogenic composition comprises an antigen having at least 80% identity with the SdrH-like polypeptide of SEQ ID NO: 8 and an antigen having at least 80% identity with LukG of SEQ ID NO: 12.

The antigens provided herein advantageously induce antibodies increasing both the uptake and the killing of S. aureus by phagocytes. Thus, the immunogenic composition of the invention advantageously comprises at least one S. aureus antigen inducing antibodies that increase both the uptake and the killing of S. aureus by phagocytes, wherein said antigen is a polypeptide having at least 80% identity with the SdrH-like polypeptide of SEQ ID NO: 8, Nuc of SEQ ID NO: 4, or LukG of SEQ ID NO: 12. Without being limited by theory, the antibody may facilitate phagocytosis or antibody dependent cellular cytotoxicity (ADCC), or both, of a S. aureus bacterium. In one case, the antigen binding portion of the opsonizing antibody binds to a target antigen, whereas the Fc portion of the opsonizing antibody binds to an Fc receptor on a phagocyte. In other cases, the antigen binding portion of the opsonizing antibody binds to a target antigen, whereas the Fc portion of the opsonizing antibody binds to an immune effector cell, e.g., via its Fc domain, thus triggering target cell lysis by the bound effector cell (e.g., monocytes, neutrophils and natural killer cells).

The immunogenic composition provided herein may comprise the S. aureus antigens in the form of separate polypeptides or in the form of one or more fusion polypeptides or both in the form of separate polypeptide(s) and fusion polypeptide(s) when multiple polypeptides are present in the immunogenic composition. As used herein, the term “fusion polypeptide” means a polypeptide created by joining two or more polypeptide sequences together. The fusion polypeptides encompassed in this invention include translation products of a chimeric gene construct that joins the DNA sequences encoding one or more antigens, or fragments or mutants thereof, with the DNA sequence encoding a second polypeptide to form a single open-reading frame. In other words, a “fusion polypeptide” is a recombinant protein of two or more proteins which are joined by a peptide bond or via several peptides.

The immunogenic composition provided herein may further comprise the same or different quantities of each component when two or more polypeptides are comprised in the immunogenic composition. As a non-limiting example, a total quantity of 50 μg of antigen may be administered per dose. It is appreciated that optimal quantity of said one or more S. aureus antigens can be determined by the artisan skilled in the art.

A further aspect of the present invention is the immunogenic composition as provided herein for use as a vaccine conferring protection against disease caused by S. aureus in a subject. The composition comprises a sufficient quantity of said one or more antigens so as to be therapeutically effective. Preferably, said vaccine is administered to a subject that does not have an existing S. aureus infection so as to induce a S. aureus-protective humoral or cellular immune response in said subject. Alternatively, said vaccine may be administered to a subject in which S. aureus infection has already occurred but that is at a sufficiently early stage such that that the immune response produced to the vaccine effectively inhibits further spread of S. aureus infection. This may notably be the case when S. aureus bacteremia (SAB) occurs, but that has not yet caused more serious infection, such as bloodstream infection or septicemia.

Said immunogenic composition or vaccine may be administered as a single dose. Alternatively, said immunogenic composition or vaccine may be administered as in multiple doses over a period of time. In particular, administration of the vaccine may be repeated as appropriate to maintain the protective effect.

Said immunogenic composition or vaccine may further comprise one or more adjuvants, which serve to enhance the magnitude, quality and/or duration of the immune response. Adjuvants for immunogenic compositions and vaccines are well-known in the art. As a non-limiting example, said adjuvants include incomplete or complete Freund's adjuvant, monoglycerides and fatty acids (e. g. a mixture of mono-olein, oleic acid, and soybean oil), mineral salts such as aluminum salts (e.g., aluminum hydroxide, aluminum phosphate, aluminum sulfate) or calcium phosphate gels, oil emulsions and surfactant based formulations (e.g., MF59 (microfluidised detergent stabilized oil-in-water emulsion), QS21 (purified saponin), AS02 [SBAS2] (oil-in-water emulsion+MPL+QS-21), MPL-SE, Montanide ISA-51 and ISA-720 (stabilised water-in-oil emulsion)), particulate adjuvants (e.g., virosomes (unilamellar liposomal vehicles incorporating influenza haemagglutinin), AS04 ([SBAS4] Al salt with MPL), ISCOMS (structured complex of saponins and lipids), polylactide co-glycolide (PLG)), natural and synthetic microbial derivatives (e.g., monophosphoryl lipid A (MPL), Detox (MPL+M. Phlei cell wall skeleton), AGP [RC-529] (synthetic acylated monosaccharide), Detox-PC, DC Chol (lipoidal immunostimulators able to self-organize into liposomes), OM-174 (lipid A derivative), CpG motifs (synthetic oligonucleotides containing immunostimulatory CpG motifs), genetically modified bacterial toxins to provide non-toxic adjuvant effects, such as modified LT and CT), endogenous human immunomodulators (e.g., hGM-CSF or hIL-12 (cytokines that can be administered either as protein or plasmid encoded), Immudaptin (C3d tandem array), MoGM-CSF, TiterMax-G, CRL- 1005, GERBU, TERamide, PSC97B, Adjumer, PG-026, GSK-I, GcMAF, B-alethine, MPC-026, Adjuvax, CpG ODN, Betafectin, Alum, and MF59) and inert vehicles, such as gold particles. Preferably, the adjuvant is a mineral salt, preferably among those listed above, more preferably aluminum hydroxide and/or aluminum phosphate. Preferably, the adjuvant is formulated as a wet gel suspension, as is the case for the Alhydrogel® and Adju-Phos® adjuvants commercialized by InvivoGen. Preferably, the ratio of antigen (Ag) to adjuvant is 0.4 to 3 mg Ag:mg aluminum (Al).

In a further aspect, the present invention relates to an immunotherapeutic composition comprising an antibody which selectively binds to at least one S. aureus antigen as provided herein (e.g., a polypeptide having at least 80% identify with the SdrH-like polypeptide, Nuc, or LukG), wherein said antibody promotes uptake and killing of S. aureus by phagocytes.

As used herein, the expression “immunotherapeutic composition” refers to a composition that comprises immune molecules (e.g., antibodies and, optionally, additional immune molecules) and that provides passive immunity. “Passive immunity” refers more particularly to any immunity conferred to a subject without administration of an antigen. It is generally temporary and short term (e.g., providing immunity for weeks or months).

As used herein, the term “antibody” refers to any polypeptide that comprises at least an antigen binding fragment or an antigen binding domain and that selectively binds a target antigen. Thus, the immunotherapeutic composition may notably include antibodies or polypeptides comprising antibody CDR domains that bind to one or more S. aureus antigens. In certain cases, it is understood that antibody binding to the target antigen is still selective despite some degree of cross-reactivity. Typically, binding between an antibody and an antigen is considered to be specific when the association constant K_{A} is higher than 10⁻⁶ M. The antibody comprised in the immunotherapeutic composition provided herein may be polyclonal, monoclonal, monospecific, polyspecific, human, humanized, single chain, chimeric, synthetic, recombinant, or any fragment of such an antibody that retains selective antigen binding, including, but not limited to, Fab, F(ab′)₂, Fv and scFv fragments. Antibodies may be whole immunoglobulin of any type (e.g., IgG, IgE, IgM, IgD, IgA and IgY), class (e.g., IgG₁, IgG₂, IgG₃, IgG₄, IgA₁ and IgA₂) or subclass. Preferably, the antibody provided herein is polyclonal. Thus, according to a preferred embodiment, the immunotherapeutic composition of the invention comprises a polyclonal antibody which selectively binds to at least one antigen as provided herein, wherein said antibody promotes uptake and killing of S. aureus by phagocytes.

The term “polyclonal antibody” as used herein more particularly refers to a mixture of antibody molecules which are capable of binding to or reacting with several different specific antigenic determinants on the same or on different antigens. Polyclonal antibodies are thus derived from different B cell lineages. The variability in antigen specificity of a polyclonal antibody is located in the variable regions of the individual antibodies constituting the polyclonal antibody, in particular in the complementarity determining regions CDR1, CDR2, and CDR3. The polyclonal antibody may be prepared by immunization of an animal, such as a horse, cow, bird, rabbit, mouse, or rat with the target antigen or portions thereof, by display (e.g., phage, yeast or ribosome display) or hybridoma techniques. Polyclonal antibody preparations may be isolated from the blood, milk, colostrum or eggs of immunized animals, and typically include antibodies that are not specific for the target antigen in addition to antibodies which are specific for the target antigen. Antibodies specific for the target antigen may be purified from the polyclonal antibody preparation or the polyclonal antibody preparation may be used without further purification. Thus, the term “polyclonal antibody” as used herein refers to both antibody preparations in which the antibody specific for the target antigen has been enriched and to preparations that are not purified. The polyclonal antibody may be provided in isolated form, in solution (e.g., animal antisera) or in host cells (e.g., hybridomas). According to a particular aspect, the immunotherapeutic composition may be a polyclonal antiserum. Numerous techniques are known to those in the art for enriching polyclonal antibodies for antibodies to specific antigens. In a certain aspect, the antibody or antibodies may be affinity purified from an animal or second subject that has been challenged with the antigen(s) provided herein. Recombinant production of highly specific polyclonal antibodies suitable for prophylactic and therapeutic administration as provided in WO 2004/061104, incorporated herein by reference in its entirety, may also be used. Recombinant polyclonal antibody (rpAb) can be purified from a production bioreactor as a single preparation without separate handling, manufacturing, purification, or characterization of the individual members constituting the recombinant polyclonal protein. Alternatively, in some cases, it may be envisaged that the polyclonal antibody is prepared by mixing multiple monoclonal antibodies.

The immunotherapeutic compositions of the present invention may be used for therapeutic purposes, e.g., for treating a subject after exposure to S. aureus. The immunotherapeutic composition may also be used prophylactically, prior to an expected or possible exposure to S. aureus (e.g., prior to orthopedic surgery, kidney dialysis). Said immunotherapeutic composition may be advantageously used for the prevention or treatment of infection by strains of S. aureus that carry the corresponding antigen(s) (e.g., “SdrH-like” polypeptide Nuc and/or LukG). Administration may be repeated as necessary to provide passive immunity over a given period of time or prior to specific events (e.g., prior to surgery).

Preferably, said prevention or treatment of infection occurs by passive immunization. Thus, according to a preferred embodiment, the immunotherapeutic composition provided herein is for use as a passive immunotherapy conferring protection against disease caused by S. aureus in a subject. In this regard, the immunotherapeutic composition may be a polyclonal composition. In a particular embodiment, the immunotherapeutic composition is a polyclonal antiserum, preferably affinity purified, from an animal which has been challenged with “SdrH-like” polypeptide, Nuc, and/or LukG antigen(s).

Preferably, the immunogenic composition of the present invention, comprising at least one S. aureus antigen or the immunotherapeutic composition comprising an antibody, preferably a polyclonal antibody, raised against said at least one S. aureus antigen, further comprises at least one pharmaceutically acceptable excipient. The term “pharmaceutically acceptable excipient” is defined herein as a component, or combination of components, that is compatible with the pharmaceutical composition, does not generate unwanted side effects in the patient, and that is generally considered to be non-toxic. A pharmaceutically acceptable excipient is most commonly implicated in facilitating administration of the composition, increasing product shelf-life or efficacy, or improving the solubility or stability of the composition. In some cases, the excipient itself may also have a therapeutic effect. The choice of said one or more excipients may furthermore depend on the desired route of administration. In the context of the present invention, the pharmaceutically acceptable excipient may notably comprise one or more diluents, adjuvants, antioxidants, preservatives, buffers and solubilizing agents. As a non-limiting example, the pharmaceutically acceptable excipient may comprise water, saline, phosphate buffered saline, sugars such as sucrose or dextrose, glycerol, ethanol, propylene glycol, polysorbate 80, poly(ethylene)glycol 300 and 400 (PEG 300 and 400), PEGylated castor oil (e.g. Cremophor EL), poloxamer 407 and 188, fat emulsions, lipids, PEGylated phospholipids, polymer matrices, biocompatible polymers, lipospheres, vesicles, liposomes, cornstarch, gelatin, lactose, sucrose, microcrystalline cellulose, kaolin, mannitol, dicalcium phosphate, calcium sulfate, sodium chloride, alginic acid, croscarmellose sodium, sodium starch glycolate, and combinations thereof.

Methods for preparing immunogenic compositions which contain antigens (i.e., polypeptides) or immunotherapeutic compositions which comprise antibodies as active ingredients are furthermore well-known in the art. Formulations can include those suitable for nasal, topical, oral (including buccal and sublingual) and/or parenteral administration. The formulations may conveniently be presented in unit dosage form. The amount of active ingredient which can be combined with a carrier material to produce a single dosage form may vary depending upon the subject and/or the particular mode of administration. The amount of active ingredient, which can be combined with a carrier material to produce a single dosage form, will generally be that amount of the compound that produces a therapeutic effect. Typically, such compositions are prepared as injectables, either as liquid solutions or suspensions, however, solid forms suitable for solution in, or suspension in, liquid prior to injection can also be prepared. The preparation can also be emulsified. The active ingredient is often mixed with excipients, such as one or more of those listed above.

As used herein, the term “S. aureus” refers to any strain of the Staphylococcus aureus species. The term encompasses laboratory strains as well as clinical isolates. According to a preferred embodiment, S. aureus is resistant to one or more antibiotics, preferably a methicillin resistant S. aureus (MRSA). The term “methicillin-resistant” indicates the lack of susceptibility of a bacterial strain to the bactericidal effects of methicillin. Resistance to methicillin is notably conferred by a mecA or mecC gene commonly located within a Staphylococcal Chromosomal Cassette (SCC). MRSA strains are also natively resistant to all agents of the beta-lactam class, with the possible exception of the so called “fifth-generation cephalosporins,” with ceftaroline and ceftobiprole being the first available agents. MRSA strains may further comprise resistance to additional antibiotics (e.g., glycopeptides, lipopeptides, mupirocin, quinolones, aminoglycosides, macrolides, rifampin, etc.). Alternatively, S. aureus may be methicillin-sensitive S. aureus (MSSA). Methicillin-sensitive strains are susceptible to the bactericidal effects of methicillin and other beta-lactams not hydrolyzed by the class A beta-lactamases commonly observed in S. aureus (notably oxacillins, cloxacillin, nafcillin, cephalosporines, carbapenems, penicillins/beta-lactam inhibitor combinations) but may comprise resistance to other antibiotics.

The expression “conferring protection against disease caused by S. aureus” as used herein refers to the prevention or the delay of the onset and/or establishment of a S. aureus associated disease or infection. As a non-limiting example, said S. aureus disease or infection may be a skin or soft tissue infection (SSTI), wound infection, bacteremia, endocarditis, pneumonia, osteomyelitis, toxic shock syndrome, infective endocarditis, folliculitis, furuncle, carbuncle, impetigo, bullous impetigo, cellulitis, botryomyosis, scalded skin syndrome, central nervous system infection, infective and inflammatory eye disease, osteomyelitis or other infections of joints or bones, respiratory tract infection, urinary tract infection, septic arthritis, septicemia, or gangrene. In particular, said S. aureus associated disease or infection may be associated with the presence of a foreign device or implant in the subject, as described herein.

The “patient” or “subject” may be as any human individual, regardless of their age. Specifically, the subject may be an adult or child. The term “adult” refers herein to an individual of at least 16 years of age. The term “child” comprises infants from 0-1 years of age and children from 1-8 years of age, 8-12 years of age, and 12-16 years of age. The term “child” further comprises neonatal infants from birth to 28 days of age and post-neonatal infants from 28 to 364 days of age. The composition may be administered to an adult or a child, including a neonatal infant. The compositions of the invention are particularly advantageous for use in the prevention or treatment of S. aureus associated disease in a subject that will undergo or that has already undergone a hospitalization for any reason, more preferably a hospitalization for cardiac or orthopedic surgery, or a dialysis treatment (e.g., kidney dialysis). In a preferred embodiment, the subject bears a foreign device or implant. As a non-limiting example, said subject may bear one or more of the following devices or implants: an intravenous catheter, a vascular prosthesis, an intravascular stent, a cerebrospinal fluid shunt, a prosthetic heart valve, a urinary catheter, a joint prosthesis, an orthopedic fixation device, a cardiac pacemaker or defibrillator, a peritoneal dialysis catheter, an intrauterine device, a biliary tract stent, a catheter for insulin administration, dentures, breast implants, contact lenses, or any other foreign device or implant. Preferably, said subject has an osteoarticular device, preferably an osteoarticular implant, more preferably a total or partial joint prosthesis, even more preferably a total or partial hip, knee, shoulder, elbow, wrist, or ankle replacement.

In a further aspect of the invention, the immunogenic or immunotherapeutic composition according to any of the embodiments provided herein is for use in the treatment of an S. aureus infection in a subject. The term “treatment” refers to a process by which the symptoms of an S. aureus infection are improved or completely eliminated. Treatment is preferably performed by internal administration of the immunogenic or immunotherapeutic composition as described herein to a subject, in combination with one or more conventional therapies, such as antibiotic therapy used in the treatment or prevention of S. aureus infection, or concomitantly with implant replacement in the case of implant failure due to S. aureus infection. Thus, according to a particular embodiment, said immunogenic or immunotherapeutic composition is for use in association with one or more antibiotics effective against a S. aureus infection.

A further aspect of the present invention concerns a method of eliciting an immune response in a subject in need thereof, comprising providing or administering the immunogenic composition described herein, for the purpose of preventing or treating a S. aureus infection. The present invention further relates to a method of preventing and/or treating a S. aureus associated disease or infection, comprising administering an immunogenic or immunotherapeutic composition according to any of the embodiments as described herein in a subject in need thereof. According to a particular embodiment, a method of conferring passive immunity to a subject in need thereof is provided herein, said method comprising the steps of (1) generating an antibody preparation using an immunogenic composition comprising at least one S. aureus antigen, wherein said antigen is a polypeptide having at least 80% identity with the SdrH-like polypeptide of SEQ ID NO: 8, Nuc of SEQ ID NO: 4, and/or LukG of SEQ ID NO: 12; and (2) administering the immunotherapeutic preparation to said subject.

Preferably, said S. aureus is an antibiotic resistant S. aureus, more preferably MRSA. Preferably, said subject bares a foreign device or implant as described herein, more preferably said subject has an osteoarticular device, preferably an osteoarticular implant, more preferably a total joint replacement prosthesis.

The present invention also comprises the use of the immunogenic or immunotherapeutic composition according to the invention for the manufacture of a medicament for raising an immune response in a subject, preferably for the prevention and/or treatment of S. aureus associated disease or infection.

The present invention also comprises the use of the immunogenic or immunotherapeutic composition according to any of the embodiments described herein in the prevention and/or treatment of S. aureus associated disease or infection.

According to a further aspect, the present invention relates to a kit comprising the immunogenic or immunotherapeutic composition as provided herein and instructions for providing or administering the immunogenic or immunotherapeutic composition described herein to a subject.

As mentioned above, in view of the high cost and duration of clinical trials, an in vitro opsonophagocytosis (OPA) assay is commonly used as a correlate of protection for assessing vaccine responses (Romero-Steiner et al., 2006), as well as for evaluating antibody functionality, in particular the ability of antibodies to promote uptake of S. aureus by professional phagocytes (Nanra et al., 2013; Fowler et al, 2013). In contrast to previous methods, which notably use polymorphonuclear neutrophils, the inventors have developed the novel OPA assay provided herein for identifying target vaccine antigens capable of antibodies promoting both uptake and killing of S. aureus.

Thus, according to a further aspect, the present invention relates to an in vitro method of identifying an antigen conferring protection against disease caused by S. aureus in a subject comprising:
a) incubating a solution comprising S. aureus with a solution comprising antibodies raised against an S. aureus antigen, preferably for one hour at 35° C., thereby obtaining a mixed suspension,
b) contacting macrophages with the mixed suspension of step a),
c) removing the mixed suspension from macrophages, and
d) assessing internalization and killing of S. aureus bacteria by said macrophages, wherein said antigen is considered to confer protection against disease caused by S. aureus when said antigen induces both increased internalization and killing of S. aureus.

Steps a), b), c) and d) of the above method are necessarily performed in the above-indicated order. Additional steps may furthermore be comprised in the method, such as culturing or diluting a solution comprising S. aureus such that the bacteria are provided at a particular density (e.g. an optical density of 1), concentrating or diluting the solution comprising antibodies, and/or diluting the mixed solution (e.g. such that the macrophages may be contacted with bacteria having a particular multiplicity of infection (MOI)), washing bacteria and/or macrophages, incubating macrophages, and the like.

While step a) is preferably performed for 1 hour at 35° C., incubation may occur for one minute to 48 hours at a temperature ranging from 2° C. to 40° C. Similarly, while step b) is preferably performed for 1 hour at 35° C., macrophages may be contacted with the mixed suspension for one minute to 48 hours at a temperature ranging from 2° C. to 38° C. Preferably, macrophages are stored at 35° C. in an atmosphere of 5% CO₂. Preferably, the mixed suspension has a MOI comprised between 10:1 and 25:1 (i.e., 10 to 25 bacteria per macrophage). The contact of the macrophage layer with S. aureus can be enhanced by centrifugation so the contact between S. aureus and the macrophages is facilitated. Centrifugation may thus advantageously reduce the duration of step b). Said step of contacting allows a proportion of S. aureus bacteria to be internalized, which may furthermore vary according to the composition of the solution comprising antibodies provided in step a). The removal of the mixed suspension in step c) may notably comprise one or more washing steps (e.g., washing the macrophages with fresh culture media or phosphate buffered saline (PBS). Indeed, this advantageously improves removal of external S. aureus bacteria. Step c) may further comprise the addition of a solution comprising antibiotics, preferably following the removal of the mixed suspension. The addition of such a solution advantageously ensures that any remaining extracellular S. aureus bacteria are killed. Thus, S. aureus should be sensitive to the antibiotic used, while macrophages are preferably unaffected. As a non-limiting example, said antibiotic is gentamicin. Gentamicin may notably be present at a concentration within the range of 50 μg/mL to 100 μg/nnL, or at a concentration equal or superior to 100 μg/nnL. As a non-limiting example, said solution comprising antibiotics is a cell culture medium, such as DMEM. Said culture medium has preferably never been used before (i.e., it is “fresh”).

Thus, according to a preferred embodiment, step c) comprises removing the mixed suspension from macrophages and adding a solution supplemented with antibiotics. More preferably, step c) comprises removing the mixed suspension from macrophages and adding fresh medium supplemented with antibiotics, thus ensuring that extracellular S. aureus bacteria are killed.

Internalization and killing of S. aureus bacteria in macrophages may be determined using methods known in the art.

As an example, internalization may be determined by quantifying bacteria according to the level of fluorescence that is observed (e.g. directly by measuring fluorescence of recombinant S. aureus bacteria expressing a fluorescent protein, or indirectly by adding one or more staining agents to fixed, permeabilized macrophages, for example BODIPY® FL Vancomycin (VMB), a fluorescent glycopeptide antibiotic that binds to the cell wall of gram positive bacteria, and measuring resulting fluorescence) according to methods known in the art (e.g. image analysis etc.). Internalization may notably be determined by comparing the level of fluorescence present 3 h after step c) in macrophages treated with a mixed suspension comprising antibodies raised against an antigen of interest versus control serum (e.g., raised against an antigen that is absent in S. aureus, such as the GFP protein) is a using image acquisition and analysis. As a particular example, image analysis may be used to determine e.g., the number of pixels positive for VMB fluorescence per macrophage in each condition. As a further example, the killing of S. aureus bacteria in step d) is assessed by comparing the quantity of bacteria internalized in macrophages at 3 hours after step c) with the quantity of bacteria internalized in macrophages at 6 hours after step c). Specifically, killing may be determined according to the level of VMB fluorescence that is observed according to the methods described herein at 3 h vs 6 h. Bacterial growth may be considered to occur when increased fluorescence was measured at 6 h as compared to 3 h. Bacterial lysis (i.e., killing) be considered to occur when a decrease in fluorescence was measured at 6 h as compared to 3 h. Such changes in fluorescence reflect the change in the amount of intracellular peptidoglycan which is, associated with bacterial growth/lysis.

The macrophages used in the method may be any macrophage cell line or isolated macrophages. Preferably, said macrophages are cultured in monolayers in classic culture conditions (i.e., in DMEM). Preferably, said macrophages are an immortalized macrophage cell line, more preferably the J774.2 cell line.

### DESCRIPTION OF THE FIGURES

FIG. 1. S. aureus uptake mediated by immune sera

S. aureus uptake (3 h post-infection) at multiplicities of infections (MOIs) of 10:1 (left panels) et 25:1 (right panels), using immune sera diluted 1/1000 (upper panels) and 1/2000 (lower panels). Average fluorescence areas values (488 nm excitation, 515 nm emission) are reported, normalized by anti-GFP antibody (value of 1). Standard deviations were calculated from the values of fluorescence areas per cell, before normalization relative to anti-GFP antibody fluorescence. Statistical significance was evaluated using Graphpad Prism on the raw data. *P-value <0.05. **P-value <0.01. Proteins tested: Pbp2a (“A”), SspA (“B”), Sak (“C”), IsaA (“D”), GlpQ (“E”), Autolysin-like protein (“F”), Nuc (“G”), Hla (“H”), LukG (“I”), LukH (“J”), IsdA (“K”), IsdB (“M”), SdrD (as two partial polypeptides: “N” and “Nb”), ClfA (as two partial polypeptides: “O” and “Ob”), MntC (“1”), SdrH-like polypeptide (“2”), Lip2 (“3”), putative protein (“4”), Atl (“5”), and hypothetical protein (“6”). Grey: Nuc (“G”), LukG (“I”) and SdrH-like polypeptide (“2”).

As shown, only five polypeptides are associated to a significant increase of uptake in at least two different conditions: Nuc (“G”), Hla (“H”), LukG (“I”), IsdA (“K”), and SdrH-like polypeptide (“2”); note that the values observed for dilutions 1/1000 et 1/2000 are similar, indicating the lack of threshold effect.

FIG. 2. S. aureus killing

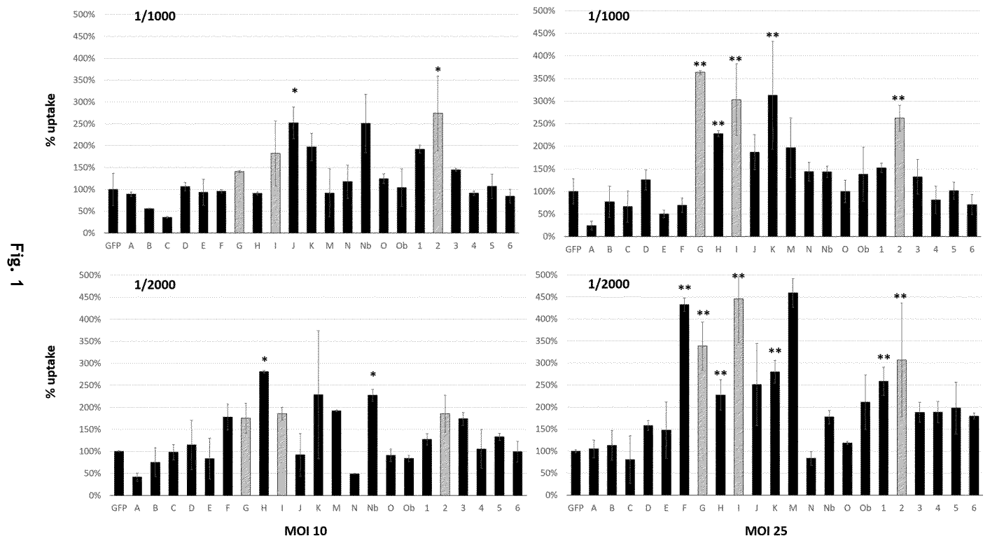

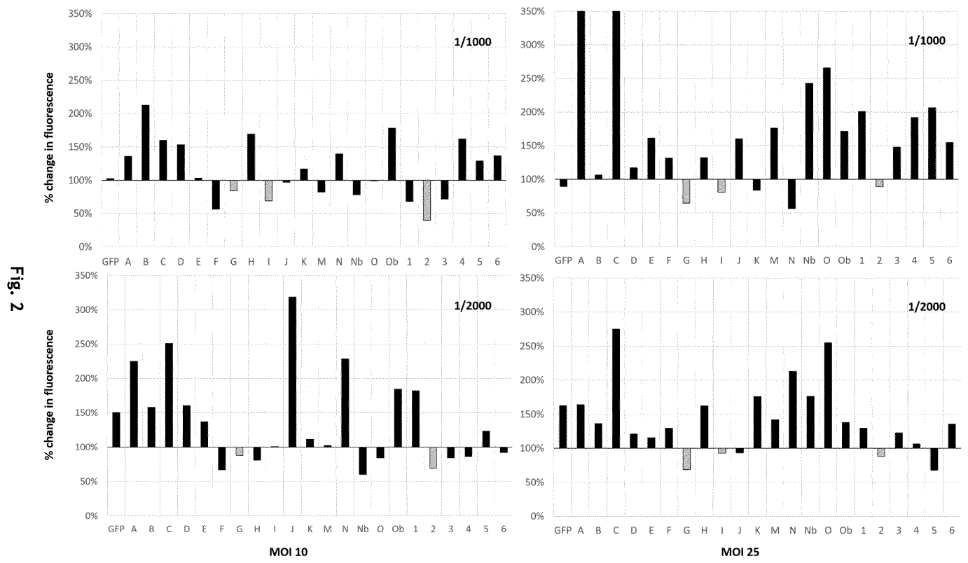

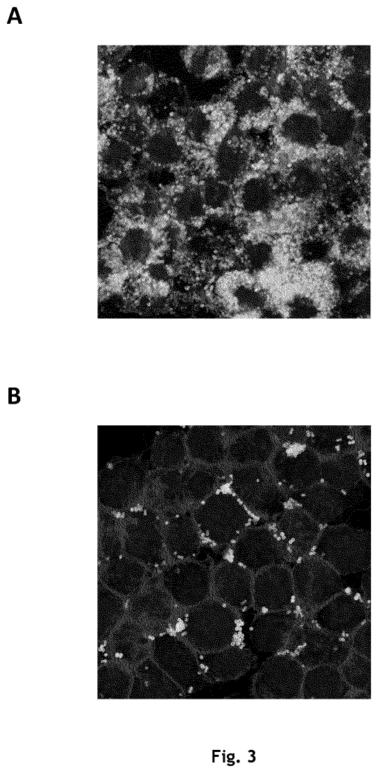

Killing of S. aureus (6 h post-infection) at MOls of 10:1 (left panels) and 25:1 (right panels), using immune sera diluted 1/1000 (upper panels) and 1/2000 (lower panels). The average fluorescence areas (excitation 488 nm, emission 515nnn) of the reported protein at the 6 h time point is normalized here to the value measured for the same protein at the 3 h time point (reference value of 1). Proteins tested are the same as those listed above (see legend of FIG. 1). Grey: Nuc (“G”), LukG (“I”), and SdrH-like polypeptide (“2”).

As shown, only three of the five antigens associated with a significant increase in

S. aureus uptake are also associated with a killing of the bacteria, in all assay conditions: Nuc (“G”), LukG (“I”), and SdrH-like polypeptide (“2”).

FIG. 3. Pictures of macrophages infected with S. aureus treated with anti-IsdB protein and anti-SdrH-like protein antisera at 6 h post-infection (MOI 1:10, serum dilution 1/1000).

With anti-IsdB (“M”) protein antiserum (panel A), myriads of bacteria (white circles) can be seen filling up cytoplasmic space; areas of cell lysis with release of extracellular bacteria can also be observed. This contrasts with the picture obtained with anti-SdrH-like polypeptide (“2”) antiserum (panel B): individual bacteria (white circles) can be enumerated; the cytoskeleton structure is preserved and the nuclei areas are preserved. Similar observations were made with anti-Nuc (“G”) protein and anti-LukG (“I”) protein antisera (data not shown).

### EXAMPLES

The following examples are included to demonstrate preferred embodiments of the invention. All subject-matter set forth or shown in the following examples and accompanying drawings is to be interpreted as illustrative and not in a limiting sense. The following examples include any alternatives, equivalents, and modifications that may be determined by a person skilled in the art.

### Example 1: Construction, Production and Purification of the S. aureus and Control Antigens

### Materials and Methods

Cloning of the Genes Coding the S. aureus Antigens of the Invention and S. aureus Control Antigens into an Expression Vector

The sequenced S. aureus strain Mu50 was used as a source of genomic DNA. DNA extraction was performed using a commercial kit (DNeasy Blood and Tissue, Qiagen Hilden, Germany). S. aureus genes of interest were amplified by polymerase chain reaction (PCR) using appropriate primers, designed with AmplifX.

Nucleotide sequences of S. aureus genes are notably as provided in SEQ ID NOs: 1, 5, 9, 13, 17, 21, 25, 29, 33, 37, 41, 45, 49, 53, 57, 61, 65, 69, 73, and 79. Cloned DNA sequences are as provided in SEQ ID NOs: 2, 6, 10, 14, 18, 22, 26, 30, 34, 38, 42, 46, 50, 54, 58, 62, 66, 70, 74, 75, 80, and 81. In particular, two different polypeptides were cloned for the sdrD and clfA genes (SEQ ID NOs: 74 and 75 for sdrD and SEQ ID NOs: 80 and 81 for clfA). DNA was purified prior enzymatic restriction with Sail and Stul (Thermo Scientific, Waltham, USA), as was the expression vector pET-6xHN-N (Clontech, Otsu, Japan) containing a poly-histidine tag. Restricted PCR products were then ligated into the vector. Resulting expression vectors of each gene were controlled by electrophoretic migration prior to transformation into chemocompetent DH10131 Escherichia coli (Thermo Scientific). Transformed bacteria were incubated 1 h at 35° C. in Luria-Bertani (LB) broth before being plated on LB agar with ampicillin (100 mg/L) and incubated overnight at 35° C. Isolated colonies were harvested and grown overnight in LB broth to amplify the clone. Vector DNA was then purified using a commercial kit (QlAprep Spin Miniprep, Qiagen). Sequencing was performed to validate each inserted gene sequence. The pET-6xHN-GFPuv vector (Clontech) was used for expressing the green fluorescent protein (GFP, SEQ ID NOs: 85 and 86 for cloned DNA and amino acid sequences, respectively).

### Antigen Production and Purification

Verified vectors were used to transform chemocompetent BL21 (DE3) E. coli cells (Thermo Scientific) following the same protocol as used for DH101β1 cells and isolated colonies similarly amplified. A 1/100 dilution of the overnight culture was incubated at 35° C. until the culture reached an optical density (OD) of 0.5. A solution of IPTG (1 mM final) was then added to the bacteria to induce antigen production at 35° C. until an OD of 1.2 was reached. Bacterial pellets obtained by centrifugation were lysed and the Histidine tagged proteins purified using a commercial kit (Proteus Metal Chelate, Generon, Slough, UK) and the recombinant His-tagged proteins eluted using a 10 mM imidazole solution. Eluted antigens were then concentrated using an Amicon Ultra-15 column (Merck, Darmstadt, Germany). Polypeptide sequences of said antigens are as provided in SEQ ID NOs: 3, 7, 11, 15, 19, 23, 27, 31, 35, 39, 43, 47, 51, 55, 59, 63, 67, 71, 76, and 82. Cloned polypeptide sequences, all of which further comprise an N-terminal His tag, are as provided in SEQ ID NOs: 4, 8, 12, 16, 20, 24, 28, 32, 36, 10, 44, 48, 52, 56, 60, 64, 68, 72, 77, 78, 83, and 84.

### Antigen Characterization

Characterization of the purified antigens was performed by sodium dodecyl sulfate-polyacrylamide gel electrophoresis (SDS-PAGE) colored with a Coomassie solution to evaluate the size, integrity and purity of the recombinant antigen. The concentration of the purified antigen solutions was determined using Bradford's method.

### Results

A total of 20 S. aureus antigens, evaluated as 22 different polypeptides, were cloned, expressed and purified (>95% of purity), with total amounts of purified protein ranging from 1 mg to 6 mg for each recombinant protein.

Four of these proteins are included in anti-S. aureus vaccines undergoing pharmaceutical development and were used as control vaccine antigens: IsdB (described in Harro et al., 2010, Moustafa et al., 2012, Fowler et al., 2013), MntC (described in Salazar et al., 2014, Begier et al., 2017, Inoue et al., 2018), CIfA (described in Salazar et al., 2014, Begier et al., 2017, Inoue et al., 2018), and Hla alpha-toxin described in Landrum et al., 2016).

### Example 2: Production of Antibodies Against S. aureus Antigens

### Materials and Methods

Antibodies targeting S. aureus polypeptides and control antigens were obtained by immunization of specific pathogen free BALB/cJRj mice, specifically documented to have originated in a S. aureus-free environment. Mice were received when they were nine weeks-old and were acclimated one week prior to immunization. Groups of six mice per antigen were injected with a first dose of 20 μg of purified antigen with Freund's complete adjuvant, followed by two more injections at 21 and 42 days with 20 μg of antigen with Freund's incomplete adjuvant. Mice were sacrificed and the sera collected 50 days after the first injection. A first control group (n=6) was injected with GFP (non-relevant non-S. aureus antigen). A second control group (n=6) was injected with Phosphate Buffer Solution (PBS) to control for adjuvant immunogenicity.

The titer and specificity of each immune serum were verified by western-blot using purified proteins.

### Results

Each serum was evaluated with serial dilutions down to a titer of 60,000. All serum sample tested showed a positive specific band at this concentration, showing the effective immunization of all animals. Sera from the six mice immunized with the same antigen were pooled to obtain a single immune serum stock for each of the 22 S. aureus polypeptides and for GFP.

### Example 3: In vitro Evaluation of Immune Sera in a Macrophage-Based OPA Assay

### Materials and Methods

S. aureus Strains

Experiments were performed with USA300 and its spA⁻ derivative (Frédéric Laurent, Lyon).

### Macrophage Culture

Cellular assays were performed on the murine BALB/c immortalized macrophage cell line J774.2 (European Collection of Authenticated Cell Lines, Porton Down, UK). Macrophages were cultured in Dulbecco's Modified Eagle Medium (DMEM) complemented with 10% fetal bovine serum at 35° C. under a 5% CO₂ atmosphere. Cells were suspended in complemented DMEM, titrated, and seeded in culture plates 24 h prior to the assay.

### Macrophage-Based Assay

An 18 h S. aureus culture in brain-heart infusion (BHI) broth was diluted 1/100 in fresh medium and incubated at 37° C. until the culture reached an OD of 1. Immune sera diluted 1/1000 or 1/2000 were added and allowed to bind to the bacterial surface for 1 h at 35° C. Serum-treated bacteria were then added to the titrated J774.2 cell monolayers at multiplicities of infection (MOI) of 10:1 (10 bacteria per cell) and 25:1 (25 bacteria per cell). After incubation for 1 h at 35° C. under a 5% CO₂ atmosphere, wells were emptied of medium and gently washed with PBS before adding fresh DMEM with gentamicin. At appropriate times (see below: bacterial uptake, 3 h post-infection; bacterial killing, 6 h post-infection), J774.2 cells were washed with PBS, fixed with PFA 4% for 5 minutes, and then permeabilized with 0.1% Triton X100 for 5 min. Fixed cells were dyed for 30 min with Hoechst 33342 (Thermo Scientific), Phalloidin-ATTO 655 (Sigma-Aldrich, Saint-Louis, USA) and BODIPY® FL Vancomycin (VMB) (Invitrogen, Carlsbad, USA), and were sealed using glass coverslips. Images were acquired using a Leica SP8 confocal microscope and analyzed using ImageJ software (National Institute of Health, Bethesda, USA).

### Evaluation of Bacterial Uptake and Killing

The uptake of serum-treated bacteria was evaluated at 3 h post-infection by comparing the number of pixels with VMB fluorescence (bacterial cell wall quantification) per J774.2 cell for each antigen specific serum to the number of pixels with VMB fluorescence per J774.2 cell for the non-relevant control serum (anti-GFP). To evaluate the outcome of internalized bacteria, the area of VMB at 3 h post-infection and 6 h post-infection were compared. Bacterial growth was called when an increase of fluorescence was measured, reflecting an increase in the amount of intracellular peptidoglycan. Bacterial lysis (“killing”) was called when a decrease in the amount of intracellular peptidoglycan was measured, reflecting a decrease in the amount of intracellular peptidoglycan.

### Results

The evaluation of bacterial uptake evaluated 3 hours after infection at MOI of 10:1 and 25:1 following S. aureus incubation with two antibody dilutions (1:1000, 1:2000) is presented here, featuring an anti-GFP control serum in each experiment for normalization. Five antigens show markedly different behaviors with a significant increase in the internalization of S. aureus bacteria in at least two conditions: proteins the SdrH-like polypeptide (“2”), Nuc (“G”), and LukG (“I”), Hla (“H”), and IsdA (“K”) (FIG. 1). Intracellular bacterial clearance and growth was evaluated by comparing the areas of VMB fluorescence 6 hours after infection to the areas of fluorescence observed 3 hours after infection. Among the five antigens previously shown to be associated with significant bacterial uptake, three were associated with bacterial killing in all experimental conditions: the SdrH-like polypeptide (“2”), Nuc (“G”), and LukG (“I”) (FIG. 2). Noticeably, a number of proteins with no significant effect on uptake were associated with bacterial growth enhancement (“facilitating” effect of immune sera) (see for example, proteins Pbp2a (“A”) and Sak (“C”) in FIG. 2, at a MOI of 25:1 and with a serum dilution of 1/1000). Bacterial growth was particularly intense with anti-IsdB protein sera and resulted in the destruction of the macrophage monolayer (FIG. 3), leading to underestimating the load of intracellular bacteria (compare FIGS. 1 Et 2 with FIG. 3).

### Example 4: Establishment of an in vivo Model of Systemic S. aureus Infection in Mice

Previous studies have shown that BALB/c mice are highly susceptible to blood-borne S. aureus infection, due to the inability of this mouse strain to limit bacterial growth in the kidneys (von Köckritz-Bliclwede et al., 2008). However, as the course of infection may differ among S. aureus strains according to their virulence repertoire, we first determined which dose of S. aureus USA300 led to non-lethal kidney infection.

### Materials and Methods

S. aureus strains

Experiments were performed with S. aureus strain USA300.

### Mice

Female BALB/c mice were purchased from Janvier Labs (Le Genest Saint Isle, France). Mice were received when they were six weeks-old and were acclimatized one week prior to immunization. Animal experiments were performed according to institutional and national ethical guidelines (Agreement APAFIS #26827).

Mouse Model of Systemic S. aureus Infection

Mice were anaesthetized by intraperitoneal administration of ketamine/xylazine (50/10 mg/kg) and were inoculated with 10⁹, 10⁷ or 10⁵ CFU of USA300 by retro-orbital sinus injection under a volume of 1004. Mice were euthanized 3 hours and 24 hours after infection. Spleen and kidneys were harvested, homogenized, and serial dilutions were plated on Mueller Hinton 2 agar plates. CFUs were enumerated after 24 hours of incubation at 37° C. (minimal detection limit: 2.69 log₁₀ CFU per organ).

### Results

The dose of 10⁹ CFU caused the death of 100% of animals before the 24 h post-challenge time-point while 10⁵ CFU did not allow the establishment of infection in the kidneys (no bacteria detected at 3 h and 24 h post-challenge) (Table 2). Thus, 10⁷ CFU was the only dose to be non-lethal and to be associated with the infection of kidneys. As expected, bacterial burden was similar at 3 h and 24 h post-challenge in the spleen, suggesting infection control, while bacterial growth was dramatically increased in the kidneys (CFU differential of ca 4 log₁₀ between 3 h and 24 h post-challenge) (Table 2).

TABLE 2
CFU counts at 3 h and 24 h post-challenge in non-immunized animals.

| | Mean number of CFUs per organ, in log₁₀ª | | | |
|---|---|---|---|---|
| | Spleen | | Kidneys | |
| USA300 dose | 3 h | 24 h | 3 h | 24 h |
| 10⁹ CFU | 6.70 | †^{b} | 6.68 | †^{b} |
| 10⁷ CFU | 5.71 | 5.58 | 2.69 | 6.08 |
| 10⁵ CFU | 3.66 | ND | ND | ND |

ªGroups of four animals per organ and at each time-point.
^{b}All animals died before the 24 h post-challenge time-point.
^{c}Not detectable (minimal detection limit, 2.69 log₁₀ CFU).

### Example 5: Evaluation of the Protective Effect of the SdrH-like Polypeptide Versus Negative Control in a Mouse Model of Systemic S. aureus Infection

BALB/c mice have been shown to be able to control S. aureus infection by developing a strong Th2 response (Nippe et al., 2011). We previously showed in the OPA assay that sera directed against the SdrH-like polypeptide, Nuc, or LukG enhanced the killing of S. aureus by phagocytes (see Example 3). We therefore studied whether the vaccination of BALB/c mice with one of these three antigens, the SdrH-like polypeptide (“SdrH-like”), may allow for an improved control of kidney infection in this model of systemic infection.

### Materials and Methods

### Production and Purification of SdrH-like, Adjuvants

SdrH-like was produced and purified as described in Example 1. Adjuvants (aluminum hydroxide gel (Alhydrogel®) and aluminum phosphate gel (Adju-Phos®); InVivoGen, CA, USA) were used according the manufacturer's recommendations.

### Vaccination Protocol and End-Point Analysis

Mice were immunized intramuscularly once a week for 3 weeks with 10 μg of purified SdrH-like (5 μg with Aluminum hydroxide gel (right thigh; volume: 50 μL) and 5 μg with Aluminum phosphate gel (left thigh, volume: 50 μL)); mice received the same quantity of adjuvants alone as a negative control.

Mice (groups of six mice per time point) were inoculated two weeks after the third immunization with a dose of 10⁷ CFU of USA300; the protocol was otherwise as described in Example 4.

### Results

As shown in Table 3, the bacterial load at 24 h post-challenge was reduced by 0.53 log₁₀ CFU in mice vaccinated with SdrH-like versus control mice. Although, kidney infection was not controlled in vaccinated mice, bacterial growth was substantially reduced (+1.53 log₁₀ CFU between 3 h and 24 h post-challenge versus+2.06 log10 CFU for control mice). As expected, vaccination had a minimal impact on spleen infection.

TABLE 3
CFU counts at 3 h and 24 h post-challenge in animals immunized
with SdrH- like versus negative control.

| | Mean number of CFUs per organ, in log₁₀ª | | | |
|---|---|---|---|---|
| Time post- | Negative control^{b} | | SdrH-like | |
| challenge | Spleen | Kidneys | Spleen | Kidneys |
| 3 h | 5.31 | 3.20 | 5.25 | 3.15 |
| 24 h | 4.05 | 5.26 | 4.33 | 4.68 |
| 3 h-24 h Δ | −1.26 | +2.06 | −0.92 | +1.53 |

ªGroups of six animals per organ and at each time-point.
^{b}Adjuvants alone.

### Example 6: Evaluation of the protective effect of SdrH-like versus staphylokinase and MntC in a mouse model of systemic S. aureus infection

SdrH-like was then compared to staphylokinase and MntC. The first comparator, staphylokinase, was chosen because sera directed against this protein were paradoxically shown to favor the intracellular growth of S. aureus in the OPA assay (see Sak, “C”, in FIGS. 1 and 2). The second comparator, MntC, was chosen because it has been shown to be a promising vaccine candidate in various animal models (Anderson et al., 2012), while it was revealed to be inferior to SdrH-like in the OPA assay (see MntC, “1”, in FIGS. 1 and 2).

Three infectious doses were tested: 10′, 3x10 6 and 10 6 CFU.

### Materials and Methods

SdrH-like, staphylokinase and MntC were produced and purified as described in Example 1. Vaccination protocol and end-point analysis were as described in Example 5, except that protective effect was evaluated using three doses: 10⁷, 3×10⁶ and 10⁶ CFU of USA300.

### Results

The course of infection in kidneys clearly differed in the mice vaccinated with SdrH-like as compared to those vaccinated with staphylokinase (Table 4); regardless of the dose of USA300, SdrH-like reduced the bacterial load in kidneys by ca 0.80 log₁₀ CFU as compared to staphylokinase (Table 5).

A similar CFU reduction (0.95 log₁₀ CFU) was observed with MntC compared to staphylokinase at the lowest dose, i.e., 10⁶ CFU (Table 5); however, the difference was much less at 10⁷ and 3×10⁶ CFU (reduction of only 0.30 to 0.39 log₁₀ CFU; Table 5).

Consistent with the above results, SdrH-like appeared to have a stronger effect than MntC on kidney infection at the two highest doses, i.e., 10⁷ and 3×10⁶ CFU (reduction of 0.45 and 0.47 log₁₀ CFU, respectively; Table 5), while similar results were found at 10⁶ CFU. Thus, the bacterial kinetics observed in the kidneys after vaccination with SdrH-like, staphylokinase and MntC paralleled the bacterial kinetics observed with these three antigens in the OPA assay.

As expected, vaccination with each of these three antigens had a minimal impact on spleen infection.

TABLE 4
CFU counts at 3 h and 24 h post-challenge in animals immunized
with SdrH-like versus staphylokinase and MntC.

| | | | Mean number of CFUs per organ, in log₁₀^{a} | | | | | |
|---|---|---|---|---|---|---|---|---|
| USA300 | | Time post- | Staphylokinase | | MntC | | SdrH-like | |
| dose | | challenge | Spleen | Kidneys | Spleen | Kidneys | Spleen | Kidneys |
| 10⁷ | CFU | 3 h | 5.28 | 3.00 | 5.09 | 3.20 | 5.24 | 3.55 |
| | | 24 h | 4.41 | 4.77 | 4.55 | 4.58 | 4.27 | 4.48 |
| | | 3 h-24 h Δ^{b} | −0.87 | +1.77 | −0.54 | +1.38 | −0.97 | +0.93 |
| 3 × 10⁶ | CFU | 3 h | 4.97 | 2.69 | 4.91 | 2.85 | 4.93 | 2.85 |
| | | 24 h | 3.79 | 4.46 | 3.90 | 4.32 | 4.08 | 3.85 |
| | | 3 h-24 h Δ | −1.18 | +1.77 | −1.01 | +1.47 | −0.85 | +1.00 |
| 10⁶ | CFU | 3 h | 4.43 | 2.69 | 4.23 | 2.85 | 4.52 | 2.69 |
| | | 24 h | 3.77 | 3.90 | 3.76 | 3.11 | 3.61 | 3.09 |
| | | 3 h-24 h Δ | −0.66 | +1.21 | −0.47 | +0.26 | −0.91 | +0.40 |

^{a}Groups of six animals per organ and at each time-point.
^{b}Bacterial growth between 3 h and 24 h is indicated by “+”, bacterial killing by “−”.

TABLE 5
Pairwise comparison of 3 h-24 h CFU differentials in the kidneys.

| | 3 h-24 h CFU differentials, in log₁₀ (difference)^{a} | | |
|---|---|---|---|
| USA300 | SdrH-like vs | SdrH-like vs | MntC vs |
| dose | staphylokinase | MntC | staphylokinase |
| 10⁷ CFU | +0.93 vs +1.77 | +0.93 vs +1.38 | +1.38 vs 1.77 |
| | (−0.84) | (-0.45) | (-0.39) |
| 3 × 10⁶ CFU | +1.0 vs +1.77 | +1.0 vs +1.47 | +1.47 vs 1.77 |
| | (−0.77) | (-0.47) | (-0.30) |
| 10⁶ CFU | +0.40 vs 1.21 | +0.40 vs +0.26 | +0.26 vs 1.21 |
| | (−0.81) | (+0.14) | (−0.95) |

ªIn bold, differences ≥ 0.5 log₁₀ CFU.

### Conclusions

The binding of specific antibodies to S. aureus can be beneficial to the host, as they may inhibit physiological functions of extracellular antigens, increase the uptake by immune cells, facilitate phagocytosis, and/or improve bacterial targeting to phagolysosomal compartments. More particularly, antibodies against S. aureus antigens may inhibit bacterial defense mechanisms targeting the bacterium to a favorable intracellular microenvironment, enhance the immune response by increasing the processing of the bacterium for antigen presentation, and foster bacterial clearance. However, certain antibodies have deleterious effects, enhancing bacterial virulence by inhibiting the function of determinants that are adequately recognized by the immune system and which participate in the control of the infection by the host. The humoral response generated by a vaccine candidate should preferably increase bacterial uptake for optimal antigen presentation and enhance intracellular bacterial lysis.

Sera directed against the SdrH-like polypeptide, Nuc, or LukG were surprisingly shown to both promote the internalization of S. aureus by macrophages and enhance the intracellular clearance of S. aureus following phagocytosis.

It is noteworthy that none of the antisera raised against the candidate vaccine proteins Hla, MntC, and CIfA previously developed and shown to be ineffective in clinical trials combined the two properties reported here. Moreover, the IsdB vaccine candidate showed to worsen the outcome of vaccinated patients was proven to be deleterious in the macrophage assay reported here, with acute destruction of the macrophage layer following enhanced internalization. These results further confirm the pertinence of the novel macrophage based in vitro assay provided herein in identifying antigens conferring protection against disease caused by S. aureus in a subject.

In addition, the results of the macrophage based in vitro assay were confirmed in vivo in a systemic model of S. aureus infection using BALB/c mice, which are highly susceptible to S. aureus due to their inability to limit bacterial growth in the kidneys. Indeed, of the three antigens evaluated in this model, the SdrH-like polypeptide showed the strongest inhibitory effect on bacterial growth of S. aureus in the kidneys overall, followed by MntC, in-line with kinetics observed in the macrophage assay.

### REFERENCES

Adlam et al., (1977) Effect immunization with highly purified alpha- and beta-toxins on staphylococcal mastitis in rabbits. Infect Immun. 17: 250-256.
Anderson et al., (2012) Staphylococcus aureus manganese transport protein C is a highly conserved cell surface protein that elicits protective immunity against S. aureus and Staphylococcus epidermidis. J Infect Dis. 205(11): 1688-1696.
Bubeck and Schneewind, (2008) Vaccine protection against Staphylococcus aureus pneumonia. J Exp Med. 205: 287-294.
Begier, et al., (2017) SA4Ag, a 4-antigen Staphylococcus aureus vaccine, rapidly induces high levels of bacteria-killing antibodies. Vaccine. 35(8): 1132-1139.
Capparelli et al., (2011) The Staphylococcus aureus Peptidoglycan Protects Mice against the Pathogen and Eradicates Experimentally Induced Infection. PLoS One. 6(12): e28377.
Darouiche, (2004) Treatment of infections associated with surgical implants. N Engl J Med. 350(14):1422-9.
Fitzgerald et al., (2011) A 12-year review of Staphylococcus aureus bloodstream infections in haemodialysis patients: more work to be done. J Hops Infect. 79(3):218-21.
Fowler, et al., (2013) Effect of an Investigational Vaccine for Preventing Staphylococcus aureus Infections After Cardiothoracic Surgery: A Randomized Trial. JAMA. 309(13): 1368-1378.
Gould, (2008) Clinical relevance of increasing glycopeptide MICs against Staphylococcus aureus. Int J Antimicrob Agents. 31 Suppl 2:1-9.
Harro et al., (2010) Safety and immunogenicity of a novel Staphylococcus aureus vaccine: results from the first study of the vaccine dose range in humans. Clin. Vaccine Immunol. 17(12): 1868-1874.
Inoue, et al., (2018) Safety, tolerability, and immunogenicity of a novel 4-antigen Staphylococcus aureus vaccine (SA4Ag) in healthy Japanese adults. Hum. Vaccines Immunother. 14(11): 2682-2691.
Landrum, et al., (2016) Safety and immunogenicity of a recombinant Staphylococcus aureus α-toxoid and a recombinant Panton-Valentine leukocidin subunit, in healthy adults. Hum. Vaccin Immunother. 13(4): 791-801.
McNeely et al., (2014) Mortality among recipients of the Merck V710 Staphylococcus aureus vaccine after postoperative S. aureus infections: An analysis of possible contributing host factors. Hum Vaccin Immunother. 10(12): 3513-3516
Moustafa, et al., (2012) Phase Ila Study of the Immunogenicity and Safety of the Novel Staphylococcus aureus Vaccine V710 in Adults with End-Stage Renal Disease Receiving Hemodialysis. Clin. Vaccine Immuno!. 19(9): 1509-1516.
Nanra et al., (2013) Capsular polysaccharides are an important immune evasion mechanism for Staphylococcus aureus. Hum Vaccin Immunother. 9(3): 480-487.
Needleman and Wunsch (1970) A general method applicable to the search for similarities in the amino acid sequence of two proteins. J Mol Biol. 48(3):443-53.
Nippe et al., (2011) Subcutaneous infection with S. aureus in mice reveals association of resistance with influx of neutrophils and Th2 response. J Invest Dermatol. 131: 125-132.
Salazar, et al., (2014) Staphylococcus aureus Manganese Transport Protein C (MntC) Is an Extracellular Matrix- and Plasminogen-Binding Protein. PLoS ONE. 9(11): e112730.
Von Köckritz-Blickwede et al., (2008) Immunological mechanisms underlying the genetic predisposition to severe Staphylococcus aureus infection in the mouse model. Am J Pathol. 173(6): 1657-1668.
Zorman (2013) Naturally occurring IgG antibody levels to the Staphylococcus aureus protein IsdB in humans. Hum Vaccin Immunother. 9(9):1857-64.

1-14. (canceled)

15. An immunogenic composition comprising at least one Staphylococcus aureus antigen, wherein said antigen is a polypeptide having at least 80% identity with the SdrH-like polypeptide of SEQ ID NO: 8, Nuc of SEQ ID NO: 4, or LukG of SEQ ID NO: 12.

16. The immunogenic composition of claim 15, comprising an antigen having at least 80% identity with the SdrH-like polypeptide of SEQ ID NO: 8 and an antigen having at least 80% identity with LukG of SEQ ID NO: 12.

17. The immunogenic composition of claim 15, wherein said composition comprises two or more of said S. aureus antigens in the form of separate polypeptides or in the form of one or more fusion polypeptides or both in the form of separate polypeptide(s) and fusion polypeptide(s).

18. The immunogenic composition of claim 15, further comprising a pharmaceutically acceptable excipient.

19. A method for conferring protection against a disease caused by S. aureus in a subject in need thereof, said method comprising the administration to the patient of the immunogenic composition of claim 15, as a vaccine.

20. A method to promote uptake and killing of S. aureus by phagocytes in a subject in need thereof, said method comprising the administration of an immunotherapeutic composition comprising a polyclonal antibody which selectively binds to at least one antigen a polypeptide having at least 80% identity with the SdrH-like polypeptide of SEQ ID NO: 8, Nuc of SEQ ID NO: 4, or LukG of SEQ ID NO: 12 .

21. The method of claim 20, wherein said immunotherapeutic composition further comprises a pharmaceutically acceptable excipient.

22. The method of claim 20, wherein said immunotherapeutic composition is used as a passive immunotherapy conferring protection against a disease caused by S. aureus in said subject.

23. The method of claim 19, wherein said S. aureus is a methicillin-resistant S. aureus (MRSA) or a methicillin-susceptible S. aureus (MSSA).

24. The method of claim 19, wherein said subject has an osteoarticular device.

25. The method of claim 19, wherein said subject has an osteoarticular implant.

26. The method of claim 19, wherein said subject has a total joint replacement prosthesis.

27. The immunogenic composition of claim 15, further containing one or more antibiotics that are effective against a S. aureus infection.

28. An in vitro method for identifying an antigen conferring protection against disease caused by S. aureus in a subject, said method comprising:
a) incubating a solution comprising S. aureus with a solution comprising antibodies raised against an S. aureus antigen, thereby obtaining a mixed suspension,
b) contacting macrophages with the mixed suspension of step a),
c) removing the mixed suspension from macrophages and adding fresh medium supplemented with antibiotics to kill extracellular S. aureus bacteria, and
d) assessing internalization and killing of S. aureus bacteria by said macrophages, wherein said antigen is considered to confer protection against disease caused by S. aureus when said antigen induces both increased internalization and killing of S. aureus.

29. The method of claim 28, wherein step a) is performed for one hour at 35° C.

30. The method of claim 28, wherein said macrophages are an immortalized macrophage cell line.

31. The method of claim 28, wherein said macrophages are the J774.2 cell line.

32. The method of claim 28, wherein the killing of S. aureus bacteria in step d) is assessed by comparing the quantity of bacteria internalized in macrophages 3 hours after step c) with the quantity of bacteria internalized in macrophages 6 hours after step c).

33. The method of claim 20, wherein said S. aureus is a methicillin-resistant S. aureus (MRSA) or a methicillin-susceptible S. aureus (MSSA).

34. The method of claim 20, wherein said subject has an osteoarticular device.
